SEQUENCE LISTING (15 sequences)

SEQ ID NO: 1 - protein, 184 aa, Artificial
  Synthetic
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - DNA, 720 bp, Artificial
  Synthetic
caggtgcagctggtgcagagcggcgcggaagtgaaaaaaccgggcgcgagcgtgaaagtgagctgcaaagcgagcggctatacctttaccagctatgtgatgcattgggtgcgccaggcgccgggccagggcctggaatggatgggctatattattccgtataacgatgcgaccaaatataacgaaaaatttaaaggccgcgtgaccatgacccgcgataccagcaccagcaccgtgtatatggaactgagcagcctgcgcagcgaagataccgcggtgtattattgcgcgcgctataactatgatggctattttgatgtgtggggccagggcaccctggtgaccgtgagcagcggcggcggcggcagcggcggcggcggcagcggcggcggcggcagcgaaattgtgctgacccagagcccggcgaccctgagcctgagcccgggcgaacgcgcgaccctgagctgccgcgcgagccagagcattagcgattatctgcattggtatcagcagaaaccgggccaggcgccgcgcctgctgatttattatgcgagccagagcattaccggcattccggcgcgctttagcggcagcggcagcggcaccgattttaccctgaccattagcagcctggaaccggaagattttgcggtgtattattgccagaacggccatagctttccgccgacctttggcggcggcaccaaagtggaaattaaa

SEQ ID NO: 3 - DNA, 354 bp, Artificial
  Synthetic
caggtgcagctggtgcagagcggcgcggaagtgaaaaaaccgggcgcgagcgtgaaagtgagctgcaaagcgagcggctatacctttaccagctatgtgatgcattgggtgcgccaggcgccgggccagggcctggaatggatgggctatattattccgtataacgatgcgaccaaatataacgaaaaatttaaaggccgcgtgaccatgacccgcgataccagcaccagcaccgtgtatatggaactgagcagcctgcgcagcgaagataccgcggtgtattattgcgcgcgctataactatgatggctattttgatgtgtggggccagggcaccctggtgaccgtgagcagc

SEQ ID NO: 4 - DNA, 321 bp, Artificial
  Synthetic
gaaattgtgctgacccagagcccggcgaccctgagcctgagcccgggcgaacgcgcgaccctgagctgccgcgcgagccagagcattagcgattatctgcattggtatcagcagaaaccgggccaggcgccgcgcctgctgatttattatgcgagccagagcattaccggcattccggcgcgctttagcggcagcggcagcggcaccgattttaccctgaccattagcagcctggaaccggaagattttgcggtgtattattgccagaacggccatagctttccgccgacctttggcggcggcaccaaagtggaaattaaa

SEQ ID NO: 5 - DNA, 45 bp, Artificial
  Synthetic
ggcggcggcggcagcggcggcggcggcagcggcggcggcggcagc

SEQ ID NO: 6 - protein, 240 aa, Artificial
  Synthetic
QVQLVQSGAEVKKPGASVKVSCKASGYTFTSYVMHWVRQAPGQGLEWMGYIIPYNDATKYNEKFKGRVTMTRDTSTSTVYMELSSLRSEDTAVYYCARYNYDGYFDVWGQGTLVTVSSGGGGSGGGGSGGGGSEIVLTQSPATLSLSPGERATLSCRASQSISDYLHWYQQKPGQAPRLLIYYASQSITGIPARFSGSGSGTDFTLTISSLEPEDFAVYYCQNGHSFPPTFGGGTKVEIK

SEQ ID NO: 7 - protein, 118 aa, Artificial
  Synthetic
QVQLVQSGAEVKKPGASVKVSCKASGYTFTSYVMHWVRQAPGQGLEWMGYIIPYNDATKYNEKFKGRVTMTRDTSTSTVYMELSSLRSEDTAVYYCARYNYDGYFDVWGQGTLVTVSS

SEQ ID NO: 8 - protein, 107 aa, Artificial
  Synthetic
EIVLTQSPATLSLSPGERATLSCRASQSISDYLHWYQQKPGQAPRLLIYYASQSITGIPARFSGSGSGTDFTLTISSLEPEDFAVYYCQNGHSFPPTFGGGTKVEIK

SEQ ID NO: 9 - protein, 15 aa, Artificial
  Synthetic
GGGGSGGGGSGGGGS

SEQ ID NO: 10 - DNA, 69 bp, Artificial
  Synthetic
atggccttaccagtgaccgccttgctcctgccgctggccttgctgctccacgccgccaggccggctagc

SEQ ID NO: 11 - DNA, 720 bp, Artificial; identical to SEQ ID NO: 2
  Synthetic

SEQ ID NO: 12 - DNA, 147 bp, Artificial
  Synthetic
aagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagccc

SEQ ID NO: 13 - DNA, 204 bp, Artificial
  Synthetic
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 14 - DNA, 342 bp, Artificial
  Synthetic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatag

SEQ ID NO: 15 - protein, 496 aa, Artificial
  Synthetic
MALPVTALLLPLALLLHAARPASQVQLVQSGAEVKKPGASVKVSCKASGYTFTSYVMHWVRQAPGQGLEWMGYIIPYNDATKYNEKFKGRVTMTRDTSTSTVYMELSSLRSEDTAVYYCARYNYDGYFDVWGQGTLVTVSSGGGGSGGGGSGGGGSEIVLTQSPATLSLSPGERATLSCRASQSISDYLHWYQQKPGQAPRLLIYYASQSITGIPARFSGSGSGTDFTLTISSLEPEDFAVYYCQNGHSFPPTFGGGTKVEIKLEKPTTTPAPRPPTPAPTIASQPLSLRPEASRPAAGGAVHTRGLDFASDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRS

(19) United States
(12) Patent Application Publication
(10) Pub. No.: US 2020/0190163 A1
(43) Pub. Date: Jun. 18, 2020

(54) HUMANIZED BCMA-CAR-T CELLS

(71) Applicants: Lijun Wu, Albany, CA (US); Vita Golubovskaya, Pinole, CA (US); Effie Ho, Green Brook, NJ (US)

(72) Inventors: Lijun Wu, Albany, CA (US); Vita Golubovskaya, Pinole, CA (US); Effie Ho, Green Brook, NJ (US)

(21) Appl. No.: 16/664,789

(22) Filed: Oct. 25, 2019

Related U.S. Application Data
(60) Provisional application No. 62/750,823, filed on Oct. 26, 2018.

(51) Int. Cl. C07K 14/715

(52) U.S. Cl. CPC C07K 14/7151; C07K 2317/622; C07K 2319/30; C07K 2319/03; C07K 2319/33; C07K 2317/24

(57) ABSTRACT

The present invention is directed to a humanized BCMA single-chain variable fragment (scFv), comprising V_{H} having the amino acid sequence of SEQ ID NO: 7 and V_{L} having the amino acid sequence of SEQ ID NO: 8. The present invention is also directed to a BCMA chimeric antigen receptor fusion protein comprising from N-terminus to C-terminus: (i) a single-chain variable fragment (scFv) of the present invention, (ii) a transmembrane domain, (iii) at least one co-stimulatory domains, and (iv) an activating domain. This humanized BCMA-CAR-T cells have specific killing activity with secretion of cytokine IFN-γ in CAR-T cells in vitro and in vivo.

This application claims the priority benefit under 35 U.S.C. section 119 of U.S. provisional patent application No. 62/750,823 entitled “Humanized BCMA-Car-T Cells”, filed on Oct. 26, 2018; which is in its entirety incorporated by reference herein.

### FIELD OF THE INVENTION

The invention relates to the field of biomedicine and specifically methods useful for cancer therapy. In certain embodiments, the invention relate to methods and compositions for carrying out cellular immunotherapy. More specifically, the present invention relates to novel humanized BCMA-CAR-T Cells specifically decreasing multiple myeloma tumor growth, which are useful in the field of adoptive immunity gene therapy for tumors. Embodiments of the invention include at least the fields of immunology, cell biology, molecular biology, and medicine, including cancer medicine. In particular embodiments, the invention relate to methods and compositions for carrying out cellular immunotherapy.

The invention also relates to isolated antibodies, or fragments or derivatives thereof, including but not limited to chimeric antigen receptors (CARs) and cells comprising CARs, which bind to antigens present in tumors, and methods of use therefor.

### BACKGROUND OF THE INVENTION

Cancer is one of the deadliest threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult. One of the difficulties in modern cancer treatments is the amount of time that elapses between a biopsy and the diagnosis of cancer, and effective treatment of the patient. During this time, a patient's tumor may grow unimpeded, such that the disease has progressed further before treatment is applied. This negatively affects the prognosis and outcome of the cancer.

Chimeric Antigen Receptors (CARs) are hybrid molecules comprising three essential units: (1) an extracellular antigen-binding motif, (2) linking/transmembrane motifs, and (3) intracellular T-cell signaling motifs. The antigen-binding motif of a CAR is commonly fashioned after a single chain Fragment variable (scFv), the minimal binding domain of an immunoglobulin (Ig) molecule. Alternate antigen-binding motifs, such as receptor ligands (i.e., IL-13 has been engineered to bind tumor expressed IL-13 receptor), intact immune receptors, library-derived peptides, and innate immune system effector molecules (such as NKG2D) also have been engineered. Alternate cell targets for CAR expression (such as NK or gamma-delta T cells) are also under development. There remains significant work with regard to defining the most active T-cell population to transduce with CAR vectors, determining the optimal culture and expansion techniques, and defining the molecular details of the CAR protein structure itself.

The linking motifs of a CAR can be a relatively stable structural domain, such as the constant domain of IgG, or designed to be an extended flexible linker. Structural motifs, such as those derived from IgG constant domains, can be used to extend the scFv binding domain away from the T-cell plasma membrane surface. This may be important for some tumor targets where the binding domain is particularly close to the tumor cell surface membrane (such as for the disialoganglioside GD2; Orentas et al., unpublished observations). To date, the signaling motifs used in CARs always include the CD3-ξ chain because this core motif is the key signal for T cell activation. The first reported second-generation CARs featured CD28 signaling domains and the CD28 transmembrane sequence. This motif was used in third-generation CARs containing CD137 (4-1BB) signaling motifs as well. With the advent of new technology, the activation of T cells with beads linked to anti-CD3 and anti-CD28 antibody, the presence of the canonical “signal 2” from CD28 was no longer required to be encoded by the CAR itself. Using bead activation, third-generation vectors were found to be not superior to second-generation vectors in in vitro assays, and they provided no clear benefit over second-generation vectors in mouse models of leukemia. This is bome out by the clinical success of CD19-specific CARs that are in a second generation CD28/CD3-ξ and a CD137/CD3-ξ signaling format. In addition to CD137, other tumor necrosis factor receptor superfamily members such as OX40 also are able to provide important persistence signals in CAR-transduced T cells. Equally important are the culture conditions under which the CAR T-cell populations were cultured.

Current challenges in the more widespread and effective adaptation of CAR therapy for cancer relate to a paucity of compelling targets. Creating binders to cell surface antigens is now readily achievable, but discovering a cell surface antigen that is specific for tumor while sparing normal tissues remains a formidable challenge. One potential way to imbue greater target cell specificity to CAR-expressing T cells is to use combinatorial CAR approaches. In one system, the CD3- and CD28 signal units are split between two different CAR constructs expressed in the same cell; in another, two CARs are expressed in the same T cell, but one has a lower affinity and thus requires the alternate CAR to be engaged first for full activity of the second. A second challenge for the generation of a single scFv-based CAR as an immunotherapeutic agent is tumor cell heterogeneity. At least one group has developed a CAR strategy for glioblastoma whereby the effector cell population targets multiple antigens (HER2, IL-13Ra, EphA2) at the same time in the hope of avoiding the outgrowth of target antigen-negative populations.

T-cell-based immunotherapy has become a new frontier in synthetic biology; multiple promoters and gene products are envisioned to steer these highly potent cells to the tumor microenvironment, where T cells can both evade negative regulatory signals and mediate effective tumor killing. The elimination of unwanted T cells through the drug-induced dimerization of inducible caspase 9 constructs with AP1903 demonstrates one way in which a powerful switch that can control T-cell populations can be initiated pharmacologically. The creation of effector T-cell populations that are immune to the negative regulatory effects of transforming growth factor-.beta. by the expression of a decoy receptor further demonstrates that degree to which effector T cells can be engineered for optimal antitumor activity.

Thus, while it appears that CARs can trigger T-cell activation in a manner similar to an endogenous T-cell receptor, a major impediment to the clinical application of CAR-based technology to date has been limited in vivo expansion of CAR+ T cells, rapid disappearance of the cells after infusion, disappointing clinical activity, relapse of the underlying medical disease or condition, and the undue length of time that elapses between diagnosis and timely treatment of cancer using such CAR+ T cells.

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Genetically modifying T cells with CAR (Chimeric antigen receptor) constructs is the most common approach to design tumor-specific T cells. CAR-T cells targeting tumor-associated antigens (TAA) can be infused into patients (called adoptive cell transfer or ACT) representing an efficient immunotherapy approach [1, 2]. The advantage of CAR-T technology compared with chemotherapy or antibody is that reprogrammed engineered T cells can proliferate and persist in the patient (“a living drug”) [1, 3, 4].

CARs typically consist of a monoclonal antibody-derived single-chain variable fragment (scFv) at the N-terminal part, hinge, transmembrane domain and a number of intracellular co-activation domains: (i) CD28, (ii) CD137 (4-1BB), CD27, or other co-stimulatory domains, in tandem with an activation CD3-zeta domain. (FIG. 1) [1, 2]. The evolution of CARs went from first generation (with no co-stimulation domains) to second generation (with one co-stimulation domain) to third generation CAR (with several co-stimulation domains). Generating CARs with two costimulatory domains (the so-called 3^{rd} generation CAR) have led to increased cytolytic CAR-T cell activity, improved persistence of CAR-T cells leading to its augmented antitumor activity.

In FIG. 1, the structures of CAR are illustrated. On the left panel—the structure of first generation (no costimulatory domains), on the middle panel—second generation (one co-stimulation domain CD28 or 4-BB) and on the right panel—third generation of CAR (two or several co-stimulation domains) are shown. The Figure is from Golubovskaya, Wu, Cancers, 2016 [6].

### BCMA

B cell maturation antigen (BCMA) is a cell surface receptor, also known as CD269 and tumor necrosis factor receptor superfamily member 17 (TNFRSF17), that is encoded by TNFRSF17 gene. This receptor is expressed mainly in mature B lymphocytes and in most cases overexpressed in multiple myeloma (MM) [3]. Current therapies to target BCMA in MM include monoclonal antibodies, bi-specific antibodies and T cellular immunotherapies, CAR-T therapies [3],[4].

### BCMA Structure and Signaling

The human BCMA protein consists of 184 amino-acids: 1-54-extracellular domain; 55-77-transmembrane domain; 78-184-cytoplasmic domain. The amino-acid sequence (SEQ ID NO: 1) of BCMA is shown on FIG. 2. BCMA lacks signaling peptide and resembles other receptors BAFF Receptor and transmembrane activator and cyclophilin ligand interactor and calcium modulator (TACT) [4]. These receptors play major role in B cell maturation and differentiation into plasma cells. Their ligands include BAFF and APRIL which expression is increase in MM patients [4].

Monoclonal antibodies target receptor-ligand interactions, and CAR-T cell therapy binds BCMA and kill MM cells. BCMA also interacts with TRAF1,2,3,5 and 6. This invention is based on humanized BCMA-CAR-T cells targeting BCMA in MM. The advantage of humanized BCMA-CAR-T cells it has humanized BCMA scFv that is less immunogenic than mouse ScFv to humans because it has human sequences in ScFV. In FIG. 2, the amino-acid sequence (SEQ ID NO: 1) of BCMA is shown with the extracellular domain underlined.

A strong medical demand exists for the chimeric antigen receptor (CAR)-T cell product described herein. Firstly, multiple myeloma is an incurable B cell non-Hodgkin lymphoma (B-NHL) which is derived from a malignantly transformed plasma cell clone. As a peculiarity, tumor cells localize predominantly to the bone marrow. This disease is the most frequent tumor of bone and bone marrow, has a 10-year survival rate of 50% among intensely treated younger patients, and is responsible for 2% of annual deaths from cancer. The incidence rate is 5/100,000, and the median age at diagnosis is 70 years, indicating that in many patients' co-morbidities exist that preclude intense and prolonged chemotherapies. The standard of care is chemotherapy, either alone or in combination with autologous stem cell transplantation, immunomodulatory drugs, local irradiation, proteasome inhibitors, and for very few patients allogeneic stem cell transplantation is applicable. Despite intense treatments with the aforementioned modalities, the disease usually relapses and after multiple lines of therapies, secondary resistance develops.

Secondly, the much larger group of classical B-NHL contain diverse entities of neoplasias derived from B lymphocytes that usually home to secondary lymphatic organs such as diffuse large B cell lymphoma (DLBCL), follicular lymphoma (FL), and a subgroup of chronic lymphocytic leukemia (CLL). While the total incidence rate of all NHL is about 10-12/100,000 (>85% of B cell origin), most of them are diseases of adults with a substantial increase in the elderly. The demographic development would predict that total numbers will increase due to aging of Western societies. Clinically, B-NHL are heterogeneous and can be distinguished by an aggressive and indolent course. Substantial progress has been made over the last 15 years in the treatment of B-NHL, the standard of care is combined antibody/chemotherapy, either alone or in combination with autologous stem cell transplantation, immunomodulatory drugs, irradiation, proteasome inhibitors, signaling pathway inhibitors, and for very few patients allogeneic stem cell transplantation applies. Because in many B-NHL entities median age at diagnosis is >55-60 years, co-morbidities also exist that preclude intense and extended chemotherapies or even allogeneic bone marrow transplantations.

The advent of adoptive CAR-T cell therapies targeted at the broadly expressed CD19 antigen on lymphoma B cells has made it possible to overcome these limitations and currently, about 20 CD19 CAR-T cell studies are registered at the FDA for the treatment of B-NHL and B-ALL. Although major breakthroughs were already achieved in clinical trials on CLL in 2011 and on B-ALL in 2013, to the best knowledge of the inventors permission to use identical CD19 CAR-products in Germany has been granted only very recently by biomedical companies. In other EU countries (e.g. Austria), clinical trials using CD19 CAR T-cells are also under way.

More importantly, in anti-CD19 antibody or CAR-T cell therapies directed against B-NHL resistance occurs due to antigen loss. Because treatment resistance is observed after multiple lines of chemo-/immunotherapy, alternative target structures are urgently warranted.

For the indication multiple myeloma, two anti BCMA-CAR products have been described previously and have entered phase I clinical studies. These studies do not prove anti-BCMA CAR applicability to B-NHL. Regarding B-NHL, anti-BCMA targeted therapies represent possible alternatives, in particular when anti-CD19 CARs have failed. Other immunotherapy strategies targeted at multiple myeloma and tested in clinical studies are anti-CD19 CARs, NY-ESO1 and MAGE-Al-directed, TCR-transduced T cells. In stark contrast to BCMA as tumor target, frequencies of eligible patients are far lower because these target antigens are expressed in less than 10% of the cases. Other targeted therapies include anti-CD38 and anti-SLAMF7 antibodies, conceptually these therapies are completely different because antibodies are not self-sustained, do not form memory and to our knowledge, are not yet proven to mediate sufficient tumor eradication.

Although a number of potential alternative therapies are in development, a significant need remains for providing effective means for addressing medical disorders associated with the presence of pathogenic B cells, in particular multiple myeloma, non-Hodgkin's lymphoma or autoantibody-dependent autoimmune diseases.

### BRIEF DESCRIPTION OF THE DRAWINGS

FIG. 1 illustrates the structures of CAR.

FIG. 2 shows the amino-acid sequence of BCMA protein.

FIG. 3 illustrates the structure of the humanized BCMA CAR construct.

FIG. 3A is another schematic of the humanized BCMA CAR construct.

FIG. 4 describes the humanized BCMA-CAR construct detected by FACS analysis with fluorescently labeled recombinant BCMA protein.

FIGS. 5A and 5B shows that humanized BCMA-CAR-T cells kill CHO-BCMA cells but not CHO cells.

FIG. 6 describes that humanized BCMA-CAR-T cells secreted high level of IFN-gamma with CHO-BCMA-positive cells but not with BCMA-negative CHO control cells.

FIG. 7 illustrates that humanized BCMA-CAR-T cells secreted high level of IFN-gamma against multiple myeloma cells but not against BCMA-negative K562 control cells. Detection of BCMA in multiple myeloma cell line but not in leukemia or other cancer cell lines.

FIGS. 8A-8D show that humanized BCMA-CAR-T cells significantly decreased RPMI8226 xenograft tumor growth. FIG. 8A illustrates that humanized BCMA-CAR-T cells significantly decreased RPMI8226 tumor volume. CAR-T cells were injected at day 7 and 20 by i.v 1×10̂7 cells/mice. Bars show average tumor volume+/−standard errors. *p<0.05, BCMA vs Mock. FIG. 8B are representative images of tumors treated with Mock control and Humanized BCMA-CAR-T cells. FIG. 8C show that BCMA-CAR-T cells are detected in BCMA-CAR-T cell-treated mice by FACS with fluorescently-labelled BCMA recombinant protein. FIG. 8D shows that humanized BCMA-CAR-T cells did not decreased mouse body weight. Bars show average mice body weight+/−standard deviations.

FIG. 9 illustrates the expansion of humanized BCMA CAR-T cells over the culture period. The control culture was identical to the CAR-T culture but was not transduced with virus, and thus contains only human T cells.

FIG. 10 describes the flow cytometric analysis of BCMA protein binding to humanized BCMA CAR-T cells and control T cells on days 8 and 13 of culture. An anti-CD3 antibody was included on day 13 (Y-axis).

FIG. 11 shows the real-time analysis of humanized BCMA CAR-T cell or control T cell cytolytic activity. CHO and CHO-BCMA cells were cultured for ˜23 hours, then huBCMA CAR-T cells or control T cells were added (vertical line).

FIG. 12 illustrates the levels of IFN-γ produced by humanized BCMA CAR-T cells and control T cells during the RTCA assay. Upon assay completion, the culture medium was centrifuged to remove cells and analyzed by ELISA.

FIG. 13 describes levels of IFN-γ produced by humanized BCMA CAR-T cells and control T cells during overnight culture with BCMA-positive myeloma cells RPMI8226 and MM1S or BCMA-negative K562 cells.

FIG. 14 illustrates the percentages of humanized BCMA CAR-T cells or control T cells expressing PD-1, TIGIT, TIM-3 or LAG-3 after overnight culture with BCMA-positive myeloma cells RPMI8226 and MM1S or BCMA-negative K562 cells. The cells were analyzed by flow cytometry.

FIG. 14A shows the flow cytometry plots of the experiments shown in FIG. 14.

FIG. 15 shows the expansion of huBCMA CAR-T cells and control T cells from 3 different donors.

FIGS. 16A and 16B feature the flow cytometric analysis of BCMA protein binding to huBCMA CAR-T cells and control T cells on days 8 and 13 of culture.

FIG. 17 illustrates the cytolytic activity of huBCMA CAR-T cells and control T cells for CHO and CHO-BCMA target cells.

FIGS. 18A, 18B and 18C show RTCA plots. FIG. 19 shows the levels of IFN-γ and IL-2 produced by humanized BCMA CAR-T cells or control T cells during the RTCA assay.

FIGS. 20A and 20B show the flow cytometric analysis of the PBMC and T cell subsets.

FIG. 21 illustrates the expansion of humanized BCMA CAR-T cells and control T cells over the culture period.

FIGS. 22A and 22B describe the flow cytometric analysis of BCMA protein binding to huBCMA CAR-T cells and control T cells.

FIGS. 23A and 23B show the flow cytometric analysis of humanized BCMA CAR-T cells and control T cells for CD4:CD8 ratios and differentiation subsets.

FIG. 24 features the cytolytic activity of huBCMA CAR-T cells and control T cells for CHO and CHO-BCMA target cells.

FIGS. 24A, 24B and 24C describes the RTCA plots from the experiment with T cell sub sets.

FIG. 25 illustrates the levels of IFN-γ and IL-2 produced by huBCMA CAR-T cells or control T cells during the RTCA assay.

FIG. 26 shows the expansion of humanized BCMA CAR-T cells over the culture period. The control culture was identical to the CAR-T culture but was not transduced with virus, and thus contains only human T cells.

FIGS. 27A and 27B show the flow cytometric analysis of BCMA protein binding to humanized BCMA CAR-T cells and control T cells on days 9 and 13 of culture.

FIG. 28 illustrates the flow cytometric analysis of huBCMA CAR-T cells. CAR-T cells were identified by BCMA protein binding and stained with antibodies for human CD4 and CD8 (left) or antibodies for human CD27 and CD45RO (right).

FIGS. 28A, 28B, 28C and 28D show the phenotype flow cytometry plots from the experiment with manufacturing medium.

FIG. 29 describes the flow cytometric analysis of huBCMA CAR-T cells. CAR-T cells were identified by BCMA protein binding and stained with antibodies for human PD-1 or human LAG-3.

FIG. 30 features the real-time analysis of humanized BCMA CAR-T cell or control T cell cytolytic activity. CHO and CHO-BCMA cells were cultured for ˜24 hours, then the medium was removed and huBCMA CAR-T cells or control T cells were added in medium lacking cytokines (vertical line). The Y-axis measures the impedance of the target cell monolayer, normalized to the time of CAR-T addition.

FIG. 31 shows the cytolytic activity of huBCMA CAR-T cells for CHO and CHO-BCMA target cells.

FIG. 32 describes the levels of IFN-γ produced by humanized BCMA CAR-T cells during overnight culture with BCMA-positive MM1S myeloma cells or BCMA-negative K562 cells.

FIG. 33 illustrates the flow cytometric analysis of manufactured huBCMA CAR-T cells. Cells were first plotted for binding to BCMA protein vs 7-AAD staining, and live CAR-T cells were gated. The gated cells were then plotted for CD4 vs CD8 staining and CD27 vs CD45RO staining.

FIG. 34 illustrates the levels of IFN-γ produced by manufactured huBCMA CAR-T cells and control T cells during the RTCA assay.

FIGS. 34A and 34B are the RTCA plots from the experiment with manufactured T cells.

FIG. 35 shows the analysis of cryopreserved manufactured huBCMA CAR-T cells. Left: cytolytic activity of cryopreserved manufactured huBCMA CAR-T cells and control T cells for CHO and CHO-BCMA target cells.

FIG. 36 describes the growth of RPMI8226 multiple myeloma tumors in mice.

FIG. 37 are photographs of the tumors at the end of the study.

FIG. 38 shows the weight of the mice in the tumor study.

FIG. 39 illustrates the frequencies of human T cells and BCMA CAR-T cells in mice.

FIG. 40 illustrates the gating and plotting scheme for analyzing humanized BCMA CAR-T cells in mice.

FIG. 41 shows the CD4:CD8 ratios of the human cells in the mice.

FIG. 42 describes the differentiation status of the control T cells and huBCMA CAR-T cells in the mice.

FIG. 43 features the levels of toxicology markers ALT and AST in the blood of treated mice.

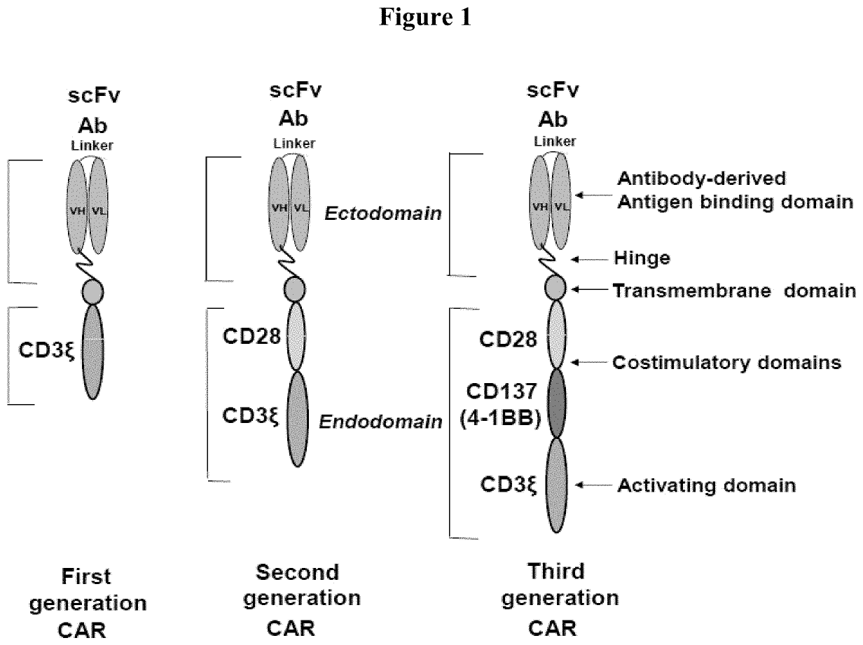

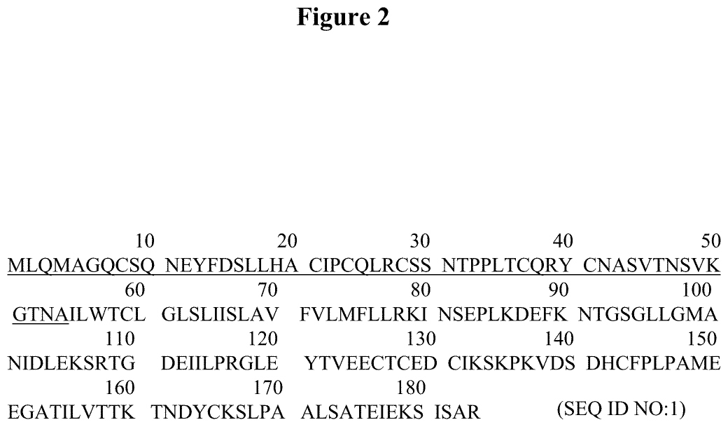

### SUMMARY OF THE INVENTION

The invention provides a single-chain variable fragment (scFv) of humanized BCMA comprising V_{H} having the amino acid of SEQ ID NO: 7, and V_{L} having the amino acid of SEQ ID NO: 8.

The invention also provides a chimeric antigen receptor (CAR) fusion protein comprising from N-terminus to C-terminus:

(i) The scFv having the amino acid sequence of SEQ ID NO: 6,

(ii) a transmembrane domain,

(iii) at least one co-stimulatory domains, and

(iv) an activating domain.

### DETAILED DESCRIPTION OF THE INVENTION

### Definitions

As used herein, a “chimeric antigen receptor (CAR)” means a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

As used herein, a “domain” means one region in a polypeptide which is folded into a particular structure independently of other regions.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for engineering an scFv are known to a person skilled in the art.

As used herein, a “tumor antigen” means a biological molecule having antigenicity, expression of which causes cancer.

The inventors have generated CAR-T cells based on novel humanized BCMA ScFv sequence specifically targeting BCMA. The inventors have produced humanized BCMA-CAR-T cells to target cancer cells overexpressing BCMA tumor antigen. The humanized BCMA-CAR-T cells of the present invention secreted high level of cytokines against multiple myeloma cancer cells and killed CHO-BCMA-positive target cells but not control CHO cells.

The present invention is directed to a humanized monoclonal anti-human BCMA antibody clone (4C8A4 or 4C8A10) comprising humanized V_{H} having the amino acid of SEQ ID NO: 5 and humanized V_{L} having the amino acid of SEQ ID NO: 6, respectively. In one embodiment, the humanized anti-human BCMA antibody is a single-chain variable fragment (scFv).

The present invention is also directed to a chimeric antigen receptor fusion protein comprising from N-terminus to C-terminus: (i) a single-chain variable fragment (scFv) against BCMA (the present invention), (ii) a transmembrane domain, (iii) at least one co-stimulatory domains, and (iv) an activating domain.

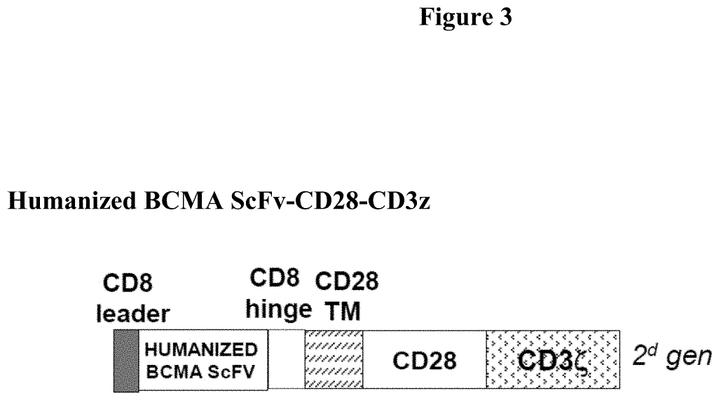

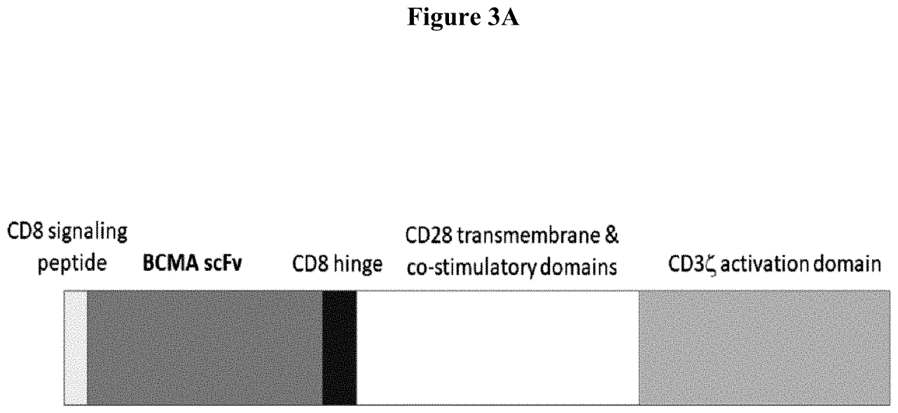

FIG. 3. The structure of humanized BCMA CAR construct. The second generation BCMA-CAR was used.

The inventors have generated humanized BCMA-ScFv-CD28-CD3-CAR-T (BCMA-CAR-T) cells against multiple myeloma cells (MM). BCMA-CAR-T cells secreted high levels of cytokines, were positive by LDH cytotoxicity assay, and by cytotoxicity assay with CHO-BCMA cells but not by CHO cells, which indicates specific killing activity of CAR-T cells against target cancer cells with their cytotoxic activity against tumor or viral antigens.

The advantages of the humanized BCMA -ScFv of the present invention include less immunogenicity to human due to humanized BCMA scFv. Thus, the BCMA antibody of the present invention is highly potent and advantageous as therapeutic agents in many clinical applications.

The present humanized BCMA ScFv can be used for immunotherapy applications: toxin/drug-conjugated Ab, monoclonal therapeutic antibody, humanization of BCMA antibody, and CAR-T cell immunotherapy.

Humanized BCMA-CAR-T cells using the present humanized BCMA ScFv can be effectively used to target BCMA antigen in BCMA-positive cancer cell lines.

Humanized BCMA-CAR-T cells can be used in combination with different chemotherapy: checkpoint inhibitors; targeted therapies, small molecule inhibitors, antibodies.

Humanized BCMA-CAR-T cells can be used clinically for BCMA-positive cancer cells.

Modifications of co-activation domains: CD28, 4-1BB and others can be used to increase its efficacy. Tag-conjugated humanized BCMA scFv can be used for CAR generation.

Humanized BCMA-CAR-T cells can be used with different safety switches: t-EGFR, RQR (Rituximab-CD34-Rituximab) and other.

Third generation CAR-T or other co-activation signaling domains can be used for the same humanized BCMA-scFv inside CAR.

The humanized BCMA CAR can be combined with CARs targeting other tumor antigens or tumor microenvironment, e.g., VEGFR-1-3, PDL-1, bi-specific antibodies with BCMA and CD3 or other antigens can be generated for therapy.

The humanized BCMA-CAR-T cells can be used against cancer stem cells that are most resistant against chemotherapy and form aggressive tumors.

A further aspect of the invention relates to a vector comprising a nucleic acid molecule as described herein, preferably a viral vector, more preferably a gamma retroviral vector.

A further aspect of the invention relates to a genetically modified immune cell comprising a nucleic acid molecule or vector as described herein, and/or expressing a CAR as described herein, wherein the immune cell is preferably selected from the group consisting of a T lymphocyte or an NK cell, more preferably cytotoxic T lymphocytes.

In an embodiment, the genetically modified immune cell comprising a nucleic acid molecule or vector as described herein, and/or expressing a CAR as described herein, is characterised in that it is CD4+ and/or CD8+ T cell, preferably a mixture of CD4+and CD8+ T cells. These T cell populations, and preferably the composition comprising both CD4+ and CD8+ transformed cells, show particularly effective cytolytic activity against various malignant B cells, such as multiple myeloma and B-NHL, preferably against those cells and/or the associated medical conditions described herein.

In another embodiment the genetically modified immune cells comprising a nucleic acid molecule or vector as described herein, and/or expressing a CAR as described herein, are CD4+ and CD8+ T cells, preferably in a ration of 1:10 to 10:1, more preferably in a ratio of 5:1 to 1:5, 2:1 to 1:2 or 1:1. Administration of BCMA-directed modified CAR-T cells expressing the CAR described herein at the ratios mentioned, preferably at a 1:1 CD4+/CD8+ ratio, lead to beneficial characteristics during treatment of the diseases mentioned herein, for example these ratios lead to improved therapeutic response and reduced toxicity.

A further aspect of the invention relates to an immune cell as described herein comprising a nucleic acid molecule or vector as described herein, and/or expressing a CAR as described herein, for use as a medicament in the treatment of a medical disorder associated with the presence of pathogenic B cells, such as a disease of plasma cells, memory B cells and/or mature B cells, in particular multiple myeloma or non-Hodgkin's lymphoma.

In one embodiment the medical use of the immune cell is characterised in that the medical disorder to be treated is multiple myeloma.

In one embodiment the medical use of the immune cell is characterised in that the medical disorder to be treated is non-Hodgkin's lymphoma.

In one embodiment the medical use of the immune cell is characterised in that the medical condition to be treated is associated with pathogenic mature B cells. To the knowledge of the inventors, no previous disclosure is apparent in the art that teaches that such mature B cells can be effectively targeted by a BCMA CAR-T, as described herein. Some of the tested tumor cell lines demonstrated in the examples below relate to mature B cells and are not necessarily of the memory type. In comparison, immature B cells would be those that give rise to acute lymphatic leukemia. The invention therefore also encompasses a method of treatment for the medical disorders disclosed herein, comprising the administration of a therapeutically effective amount of a CAR or a therapeutic agent comprising the CAR of the present invention to a subject in need of such treatment.

A further aspect of the invention relates to a pharmaceutical composition comprising the CAR or therapeutic agent comprising a CAR as described herein together with a pharmaceutically acceptable carrier.

Multiple myeloma, also referred to as plasmocytoma, is a currently incurable B cell lymphoma which is derived from a malignantly transformed plasma cell clone. This disease constitutes the most frequent tumor of bone and bone marrow, has a median life-expectancy of seven years and is responsible for 2% of annual deaths from cancer. The malignant transformation is believed to occur in germinal centers of secondary lymphoid organs at a developmental stage where B cells have completed VDJ-rearrangement and isotype switching. The median age at diagnosis is 70 years, indicating that in many patients co-morbidities exist that preclude intensive and prolonged chemo- or radiotherapies. Moreover, allogeneic bone marrow transplantations are usually excluded for this patient cohort. The disease is characterized clinically by osteolytic lesions, hypercalcemia, hematopoietic insufficiency, amyloid deposition, renal failure, excessive antibody heavy and/or light chain production, hyper viscosity, infections, bleeding disorders. The standard of care is chemotherapy, either alone or in combination with autologous stem cell transplantation, immunomodulators such as immunomodulatory drugs (IMIDs), local irradiation, proteasome inhibitors, and for a few patients allogeneic stem cell transplantation applies. Despite intensive treatments with the aforementioned modalities, the disease usually relapses and after multiple lines of therapies primary and secondary resistances develop.

The adoptive chimeric antigen receptor (CAR)-T cell therapies described herein targeted at the B cell maturation antigen (BCMA) can overcome these limitations in multiple myeloma because BCMA is highly expressed in multiple myeloma tumor cells, but not in normal B cells or precursor B cells. Secondly, in anti-CD19 antibody or anti-CD19 CAR-T cell therapies directed against B cell non-Hodgkin's lymphoma (B-NHL) resistances occur due to antigen loss. Because treatment resistance occurs after multiple lines of chemo-/immunotherapy in these B-NHLs, alternative target structures are warranted. For mature B-NHL, BCMA is a suitable target and therefore, the anti-BCMA CAR-T cells with a high affinity can be employed therapeutically even in B-NHL as specified below.

BCMA CAR-T cell transfers are selective for the tumor-associated antigen BCMA, applicable and effective even for the elderly and after multidrug resistances have appeared. They have predictable, tolerable and manageable side effects. Autologous T cells equipped with the anti-BCMA CAR have a high affinity and avidity and recognize and destroy multiple myeloma cells while sparing normal hematopoietic cells such as T cells, B cells and their bone marrow precursors; all myeloid cells and NK cells are likewise spared. Due to autologous transfer of T cells a graft-versus-host-disease cannot occur. Memory T cell formation which is important for the prevention of a relapse can develop. Due to the high affinity and avidity of the anti-BCMA CAR-T cell, even low BCMA-expressing mature B cell NHL can be recognized, allowing for T cell activation and tumor cell killing. Such mature B-NHL entities include certain stages of follicular lymphoma, diffuse large B-cell lymphoma, mantle cell lymphoma, and chronic lymphocytic leukemia.

The anti-BCMA CAR-T cell described herein is in some embodiments applicable to multiple myeloma and B-NHL patients who are not eligible for other therapies. More specifically: i) patients with multidrug resistances, ii) patients not eligible for allogeneic stem cell transplantation, iii) patients with co-morbidities that preclude further chemotherapies, iv) aged patients who do not tolerate chemotherapies, v) the CAR is applicable for salvage therapies even after progressive disease and multiple lines of other standard of care therapies have failed, vi) it is applicable even at very low antigen density on target tumor cells, where antibodies can fail, vii) a structure of the source antibody complexed with BCMA at near atomic resolution verifies its exquisite specificity, a biosafety feature not shown for other anti-BCMA CAR-T cells, and/or vii) it is applicable as a monotherapy which is not the case for antibodies.

For other anti-BCMA CAR-T cells described in the art their reactivity has only been shown for multiple myeloma cells and patients; in contrast, our anti-BCMA CAR has an unexpectedly high sensitivity even for low BCMA expressing B-NHL cell lines. Our anti-BCMA CAR confers extremely high avidity to T cells, necessary for anti-tumor efficacy. No other anti-BCMA CAR is reported to react against mature B-NHL, diffuse large B-cell lymphoma (DLBCL), defined stages of follicular lymphoma, mantle cell lymphoma, or chronic lymphocytic leukemia. The present invention demonstrates that our anti-BCMA CAR does not confer T cell-reactivity against physiological B cells, T cells, NK cells, endothelial cells, all myeloid cell lineages and their precursors. Thus, the present invention has an unprecedented low off-target reactivity on other hematopoietic tissues. In contrast to anti-CD38 CAR-T cells, our anti-BCMA CAR has no unwanted reactivity against myeloid cell precursors.

CARs contemplated herein, comprise an extracellular domain (also referred to as a binding domain or antigen-binding domain) that binds to BCMA, a transmembrane domain, and an intracellular domain, or intracellular signaling domain. Engagement of the anti-BCMA antigen binding domain of the CAR with BCMA on the surface of a target cell results in clustering of the CAR and delivers an activation stimulus to the CAR-containing cell. The main characteristic of CARs are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In various embodiments, a CAR comprises an extracellular binding domain that comprises a humanized BCMA-specific binding domain; a transmembrane domain; one or more intracellular signaling domains. In particular embodiments, a CAR comprises an extracellular binding domain that comprises a humanized anti-BCMA antigen binding fragment thereof; one or more spacer domains; a transmembrane domain; one or more intracellular signaling domains.

The “extracellular antigen-binding domain” or “extracellular binding domain” are used interchangeably and provide a CAR with the ability to specifically bind to the target antigen of interest, BCMA. The binding domain may be derived either from a natural, synthetic, semi-synthetic, or recombinant source. Preferred are scFV domains.

In certain embodiments, the CARs contemplated herein may comprise linker residues between the various domains, added for appropriate spacing and conformation of the molecule, for example a linker comprising an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. CARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids.

Illustrative examples of linkers include glycine polymers; glycine-serine polymers; glycine-alanine polymers; alanine-serine polymers; and other flexible linkers known in the art, such as the Whitlow linker. Glycine and glycine-serine polymers are relatively unstructured, and therefore may be able to serve as a neutral tether between domains of fusion proteins such as the CARs described herein.

In particular embodiments, the binding domain of the CAR is followed by one or more “spacers” or “spacer polypeptides,” which refers to the region that moves the antigen binding domain away from the effector cell surface to enable proper cell/cell contact, antigen binding and activation. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region. In one embodiment, the spacer domain comprises the CH2 and CH3 domains of IgG1 or IgG4.

The binding domain of the CAR may in some embodiments be followed by one or more “hinge domains,” which play a role in positioning the antigen binding domain away from the effector cell surface to enable proper cell/cell contact, antigen binding and activation. A CAR may comprise one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

Illustrative hinge domains suitable for use in the CARs described herein include the hinge region derived from the extracellular regions of type 1 membrane proteins such as CD8 alpha, CD4, CD28, PD1, CD 152, and CD7, which may be wild-type hinge regions from these molecules or may be altered. In another embodiment, the hinge domain comprises a PD1, CD 152, or CD8 alpha hinge region.

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from the alpha, beta or zeta chain of the T-cell receptor, CD3.epsilon., CD3.zeta., CD4, CD5, CD8 alpha, CD9, CD 16, CD22, CD27, CD28, CD33, CD37, CD45, CD64, CD80, CD86, CD 134, CD 137, CD 152, CD 154, and PD1. In one embodiment, the CARs contemplated herein comprise a TM domain derived from CD8 alpha or CD28

In particular embodiments, CARs contemplated herein comprise an intracellular signaling domain. An “intracellular signaling domain,” refers to the part of a CAR that participates in transducing the message of effective anti-BCMA CAR binding to a human BCMA polypeptide into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function.

CARs contemplated herein comprise one or more co-stimulatory signaling domains to enhance the efficacy, expansion and/or memory formation of T cells expressing CAR receptors. As used herein, the term, “co-stimulatory signaling domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen.

The genetically modified immune effector cells contemplated herein provide improved methods of adoptive immunotherapy for use in the treatment of B cell related conditions that include, but are not limited to immunoregulatory conditions and hematological malignancies.

In particular embodiments, compositions comprising immune effector cells comprising the CARs contemplated herein are used in the treatment of conditions associated with abnormal B cell activity, otherwise termed as a “medical disorder associated with the presence of pathogenic B cells”.

As used herein, “medical disorder associated with the presence of pathogenic B cells” or “B cell malignancy” refers to a medical condition, such as cancer, that forms in B cells. In particular embodiments, compositions comprising CAR-modified T cells contemplated herein are used in the treatment of hematologic malignancies, including but not limited to B cell malignancies such as, for example, multiple myeloma (MM) and non-Hodgkin's lymphoma (NHL).

In another aspect of the present invention there is provided a CAR and CAR-T according to the invention as herein described for use in the treatment of a B-cell mediated or plasma cell mediated disease or antibody mediated disease or disorder selected from Multiple Myeloma (MM), chronic lymphocytic leukemia (CLL), Non-secretory multiple myeloma, Smoldering multiple myeloma, Monoclonal gammopathy of undetermined significance (MGUS), Solitary plasmacytoma (Bone, Extramedullar), Lymphoplasmacytic lymphoma (LPL), Waldenstrom's Macroglobulinemia, Plasma cell leukemia, Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpasture's syndrome, Idiopathic thrombocytopenic purpura (ITP), Acute glomerulonephritis, Pemphigus and Pemphigoid disorders, and Epidermolysis bullosa acquisita; or any Non-Hodgkin's Lymphoma B-cell leukemia or Hodgkin's lymphoma (HL) with BCMA expression or any diseases in which patients develop neutralising antibodies to recombinant protein replacement therapy wherein said method comprises the step of administering to said patient a therapeutically effective amount of the CAR or CAR-T as described herein.

Multiple myeloma is a B cell malignancy of mature plasma cell morphology characterized by the neoplastic transformation of a single clone of these types of cells. These plasma cells proliferate in BM and may invade adjacent bone and sometimes the blood. Variant forms of multiple myeloma include overt multiple myeloma, smoldering multiple myeloma, plasma cell leukemia, non-secretory myeloma, IgD myeloma, osteosclerotic myeloma, solitary plasmacytoma of bone, and extramedullary Plasmacytoma.

Non-Hodgkin lymphoma encompasses a large group of cancers of lymphocytes (white blood cells). Non-Hodgkin lymphomas can occur at any age and are often marked by lymph nodes that are larger than normal, fever, and weight loss. Non-Hodgkin lymphomas can also present on extranodal sites, such as the central nervous system, mucosal tissues including lung, intestine, colon and gut. There are many different types of non-Hodgkin lymphoma. For example, non-Hodgkin's lymphoma can be divided into aggressive (fast-growing) and indolent (slow-growing) types. Although non-Hodgkin lymphomas can be derived from B cells and T-cells, as used herein, the term “non-Hodgkin lymphoma” and “B cell non-Hodgkin lymphoma” are used interchangeably. B cell non-Hodgkin lymphomas (NHL) include Burkitt lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, and mantle cell lymphoma. Lymphomas that occur after bone marrow or stem cell transplantation are usually B cell non-Hodgkin lymphomas.

Chronic lymphocytic leukemia (CLL) is an indolent (slow-growing) cancer that causes a slow increase in immature white blood cells called B lymphocytes, or B cells. Cancer cells spread through the blood and bone marrow, and can also affect the lymph nodes or other organs such as the liver and spleen. CLL eventually causes the bone marrow to fail. A different presentation of the disease is called small lymphocytic lymphoma and localizes mostly to secondary lymphoid organs, e.g. lymph nodes and spleen.

As used herein “treatment” or “treating,” includes any beneficial or desirable effect on the symptoms or pathology of a disease or pathological condition, and may include even minimal reductions in one or more measurable markers of the disease or condition being treated.

Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” or “prophylactic” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In one embodiment, a method of treating a B cell related condition in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

The following examples further illustrate the present invention. These examples are intended merely to be illustrative of the present invention and are not to be construed as being limiting.

### EXAMPLES

The inventors generated humanized BCMA-ScFv-CAR constructs inside lentiviral vector cloned into Xba I and Eco R I sites of lentiviral vector. pCD510-FMC63-28z lentiviral CAR construct containing the humanized BCMA ScFv-CD28-CD3ξ insert—between the Xba I and Eco RI cloning sites.

The lentiviruses were generated in 293T cells and titer was established by RT-PCR. Then equal dose of lentiviruses was used for transduction of T cells.

### Example 1

### Humanized BCMA VH And VL and scFV Sequences

The BCMA scFv was obtained by sequencing hybridoma clones 4C8A4 and 4C8A10 positive for BCMA. The structure of humanized BCMA (PMC305) scFv is: VH-linker-VL. The Linker is G4S×3. The entire sequence of PMC 305 is SEQ ID NO: 2 having the following sequence:

caggtgcagctggtgcagagcggcgcggaagtgaaaaaaccgggcgcgag
cgtgaaagtgagctgcaaagcgagcggctatacctttaccagctatgtga
tgcattgggtgcgccaggcgccgggccagggcctggaatggatgggctat
attattccgtataacgatgcgaccaaatataacgaaaaatttaaaggccg
cgtgaccatgacccgcgataccagcaccagcaccgtgtatatggaactga
gcagcctgcgcagcgaagataccgcggtgtattattgcgcgcgctataac
tatgatggctattttgatgtgtggggccagggcaccctggtgaccgtgag
cagcggcggcggcggcagcggcggcggcggcagcggcggcggcggcagcg
aaattgtgctgacccagagcccggcgaccctgagcctgagcccgggcgaa
cgcgcgaccctgagctgccgcgcgagccagagcattagcgattatctgca
ttggtatcagcagaaaccgggccaggcgccgcgcctgctgatttattatg
cgagccagagcattaccggcattccggcgcgctttagcggcagcggcagc
ggcaccgattttaccctgaccattagcagcctggaaccggaagattttgc
ggtgtattattgccagaacggccatagctttccgccgacctttggcggcg
gcaccaaagtggaaattaaa

The bold highlights the nucleotide sequence of humanized BCMA PMC305 clone: V_{H} SEQ ID NO: 3 having the sequence:

caggtgcagctggtgcagagcggcgcggaagtgaaaaaaccgggcgcgag
cgtgaaagtgagctgcaaagcgagcggctatacctttaccagctatgtga
tgcattgggtgcgccaggcgccgggccagggcctggaatggatgggctat
attattccgtataacgatgcgaccaaatataacgaaaaatttaaaggccg
cgtgaccatgacccgcgataccagcaccagcaccgtgtatatggaactga
gcagcctgcgcagcgaagataccgcggtgtattattgcgcgcgctataac
tatgatggctattttgatgtgtggggccagggcaccctggtgaccgtgag
cagc.

The underlined highlights the nucleotide sequence of V_{L} SEQ ID NO: 4 having the sequence:

gaaattgtgctgacccagagcccggcgaccctgagcctgagcccgggcga
acgcgcgaccctgagctgccgcgcgagccagagcattagcgattatctgc
attggtatcagcagaaaccgggccaggcgccgcgcctgctgatttattat
gcgagccagagcattaccggcattccggcgcgctttagcggcagcggcag
cggcaccgattttaccctgaccattagcagcctggaaccggaagattttg
cggtgtattattgccagaacggccatagctttccgccgacctttggcggc
ggcaccaaagtggaaattaaa

In between (italicized) is the nucleotide sequence SEQ ID NO: 5 encoding the 3×G4S linker 3×(GGGGS) having the sequence ggcggcggcggcagcggcggcggcggcagcggcggcggcggcagc.

The humanized BCMA (PMC305) scFv Protein has the sequence SEQ ID NO: 6 having the following sequence:

QVQLVQSGAEVKKPGASVKVSCKASGYTFTSYVMHWVRQAPGQGLEWMGY
IIPYNDATKYNEKFKGRVTMTRDTSTSTVYMELSSLRSEDTAVYYCARYN
YDGYFDVWGQGTLVTVSS G G G G S G G G G S G G G G S E
IVLTQSPATLSLSPGERATLSCRASQSISDYLHWYQQKPGQAPRLLIYYA
SQSITGIPARFSGSGSGTDFTLTISSLEPEDFAVYYCQNGHSFPPTFGGG
TKVEIK

In the protein, the bold highlights the amino acid sequence of V_{H} SEQ ID NO: 7 having the sequence:

QVQLVQSGAEVKKPGASVKVSCKASGYTFTSYVMHWVRQAPGQGLEWMGY
IIPYNDATKYNEKFKGRVTMTRDTSTSTVYMELSSLRSEDTAVYYCARYN
YDGYFDVWGQGTLVTVSS

The underlined highlights the amino sequence of V_{L} SEQ ID NO: 8 having the sequence

EIVLTQSPATLSLSPGERATLSCRASQSISDYLHWYQQKPGQAPRLLIYY
ASQSITGIPARFSGSGSGTDFTLTISSLEPEDFAVYYCQNGHSFPPTFGG
GTKVEIK

In between (italicized) is the amino acid sequence of 3×G4S linker sequence (SEQ ID NO: 9 having the sequence G G G G S G G G G S G G G G S.

### Example 2

### Humanized BCMA-CAR Sequences

The scheme of Humanized (PMC305) BCMA-CAR construct is shown on FIG. 3. We will call it humanized BCMA throughout the application. Lentiviral vector with EFla promoter was used for cloning of humanized scFv CAR sequences.

The following nucleotide sequence shows CD8 leader-Humanized BCMA ScFv-CD8 hinge-TM28-CD28-CD3ξ of the present invention. The CAR structure includes Human CD8 signaling peptide, humanized BCMA scFv (V_{H}-Linker 3×(G4S)-V_{L}), CD8 hinge, CD28 transmembrane, activation domains, CD3ξ (FIG. 3).

CD8 leader sequence-BCMA scFv (V_{H}-Linker -V_{L})-CD8 hinge. CD28 TM-CD28-CD3ξ:

CD8 leader
SEQ ID NO: 10
ATGGCCTTACCAGTGACCGCCTTGCTCCTGCCGCTGGCCTTGCTGCTCCA
CGCCGCCAGGCCGGCTAGC
Humanized BCMA, clone 4C8A4 /4C8A10 scFv
SEQ ID NO: 11
caggtgcagctggtgcagagcggcgcggaagtgaaaaaaccgggcgcgag
cgtgaaagtgagctgcaaagcgagcggctatacctttaccagctatgtga
tgcattgggtgcgccaggcgccgggccagggcctggaatggatgggctat
attattccgtataacgatgcgaccaaatataacgaaaaatttaaaggccg
cgtgaccatgacccgcgataccagcaccagcaccgtgtatatggaactga
gcagcctgcgcagcgaagataccgcggtgtattattgcgcgcgctataac
tatgatggctattttgatgtgtggggccagggcaccctggtgaccgtgag
cagcggcggcggcggcagcggcggcggcggcagcggcggcggcggcagcg
aaattgtgctgacccagagcccggcgaccctgagcctgagcccgggcgaa
cgcgcgaccctgagctgccgcgcgagccagagcattagcgattatctgca
ttggtatcagcagaaaccgggccaggcgccgcgcctgctgatttattatg
cgagccagagcattaccggcattccggcgcgctttagcggcagcggcagc
ggcaccgattttaccctgaccattagcagcctggaaccggaagattttgc
ggtgtattattgccagaacggccatagctttccgccgacctttggcggcg
gcaccaaagtggaaattaaa
XhoI restriction site Sequence
CTCGAG
CD8 hinge
SEQ ID NO: 12
AAGCCCACCACGACGCCAGCGCCGCGACCACCAACACCGGCGCCCACCAT
CGCGTCGCAGCCCCTGTCCCTGCGCCCAGAGGCGAGCCGGCCAGCGGCGG
GGGGCGCAGTGCACACGAGGGGGCTGGACTTCGCCAGTGATaagccc
CD28 TM/activation
SEQ ID NO: 13
TTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGC
TCCTGCACAGTGACTACATGAACATGACTCCCCGCCGCCCCGGGCCCACC
CGCAAGCATTACCAGCCCTATGCCCCACCACGCGACTTCGCAGCCTATCG
CTCC
CD3 zeta
SEQ ID NO: 14
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAATAG

EcoRI restriction site Sequence gaattc

Translated amino-acid sequence of humanized BCMA-CAR protein (see FIG. 3 for construct structure), SEQ ID NO: 15

(SEQ ID NO: 17)
M A L P V T A L L L P L A L L L H A A R P A S Q V
Q L V Q S G A E V K K P G A S V K V S C K A S G Y
T F T S Y V M H W V R Q A P G Q G L E W M G Y I I
P Y N D A T K Y N E K F K G R V T M T R D T S T S
T V Y M E L S S L R S E D T A V Y Y C A R Y N Y D
G Y F D V W G Q G T L V T V S S G G G G S G G G G
S G G G G S E I V L T Q S P A T L S L S P G E R A
T L S C R A S Q S I S D Y L H W Y Q Q K P G Q A P
R L L I Y Y A S Q S I T G I P A R F S G S G S G T
D F T L T I S S L E P E D F A V Y Y C Q N G H S F
P P T F G G G T K V E I K L E K P T T T P A P R P
P T P A P T I A S Q P L S L R P E A S R P A A G G
A V H T R G L D F A S D K P F W V L V V V G G V L
A C Y S L L V T V A F I I F W V R S K R S R L L H
S D Y M N Met T P R R P G P T R K H Y Q P Y A P P
R D F A A Y R S R V K F S R S A D A P A Y Q Q G Q
N Q L Y N E L N L G R R E E Y D V L D K R R G R D
P E M G G K P Q R R K N P Q E G L Y N E L Q K D K
Met A E A Y S E I G M K G E R R R G K G H D G L Y
Q G L S T A T K D T Y D A L H M Q A L P P R S

### Example 3

### Humanized BCMA-CAR-T Cells Kill Mulitple Myeloma Cells and Secrete High Level of IFN-Gamma Against BCMA-Positive Cancer Cells

We designed humanized BCMA-CAR-T cells with humanized BCMA-CAR construct as shown in FIG. 3.

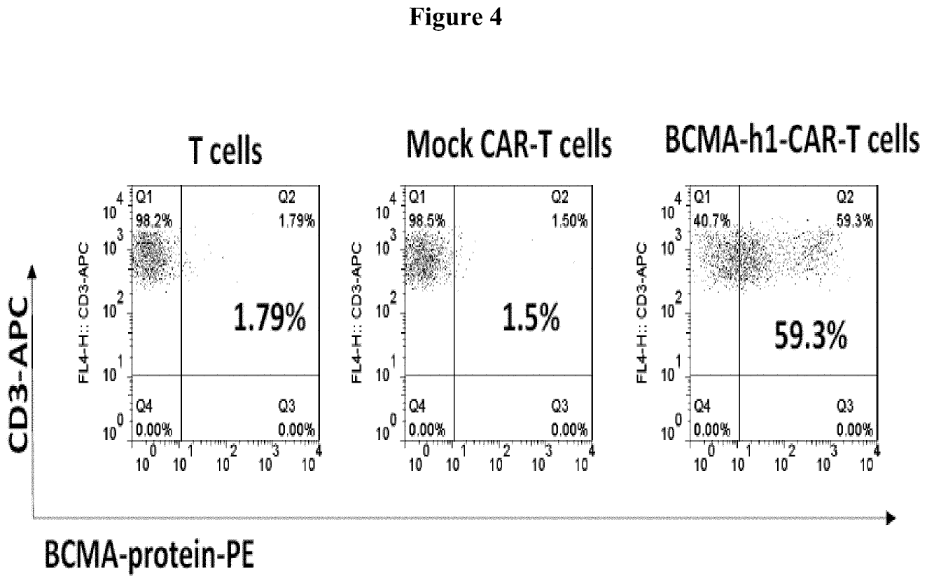

We used Mock scFv with unrelated ScFv and generated Mock-CAR-T cells as a negative control. Humanized BCMA-CAR-T cells expressed BCMA scFv as detected by FACS (FIG. 4). FIG. 4 shows the humanized BCMA-CAR construct is detected by FACS analysis with fluorescently labeled recombinant BCMA protein. Humanized BCMA-CAR-positive cells were detected after transduction of lentiviral humanized BCMA CAR into T cells.

### Example 4

### Humanized BCMA-CAR-T Cells Killed CHO-BCMA Cells but not CHO Cells

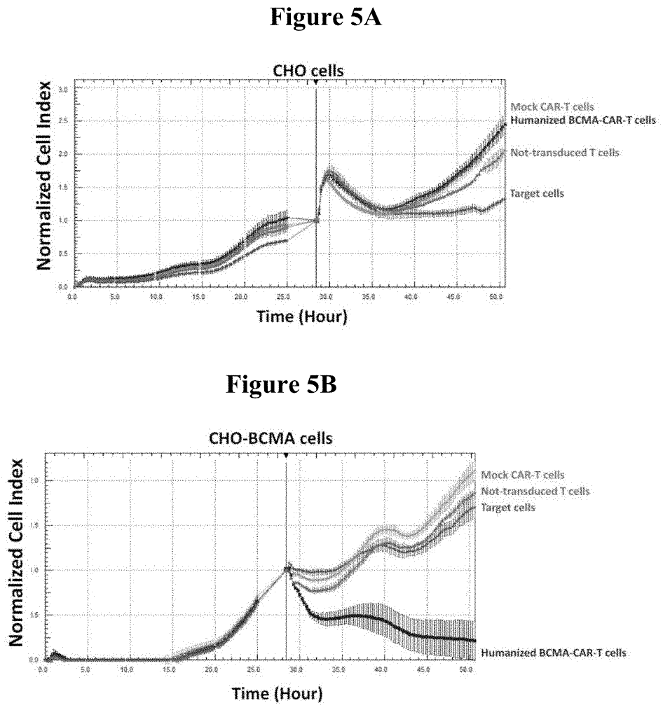

We incubated humanized BCMA-CAR-T cells with target CHO-BCMA target cells and also CHO (BCMA-negative) control cells. Humanized BCMA-CAR-T cells specifically killed CHO-BCMA cells (FIG. 5A, upper panel) but not CHO cells (FIG. 5B, lower panel). This demonstrate high specificity of humanized BCMA-CAR-T cells to targeting BCMA antigen and killing BCMA-positive cells.

FIG. 5 shows humanized BCMA-CAR-T cells kill CHO-BCMA cells but not CHO cells. XCelligence Real-time cytotoxicity assay was used for detection of humanized BCMA-CAR-T cell cytotoxicity. Normalized cell index is shown on Y-axis, and time is shown on X-axis. Upper panel: CHO-BCMA target cells. on the right: From top to bottom: Mock, T cells, Mock-CAR-T cells and humanized CAR-T cells are shown as effector cells; Lower panel CHO target cells. From top to bottom on the right, Mock CAR-T cells, Humanized BCMA CAR-T cells, T cells and target cells are shown as effector cells.

### Example 5

### Humanized CAR-T Cells Secrete IFN-Gamma Against Target CHO-BCMA Cells Significantly but not Against CHO Cells

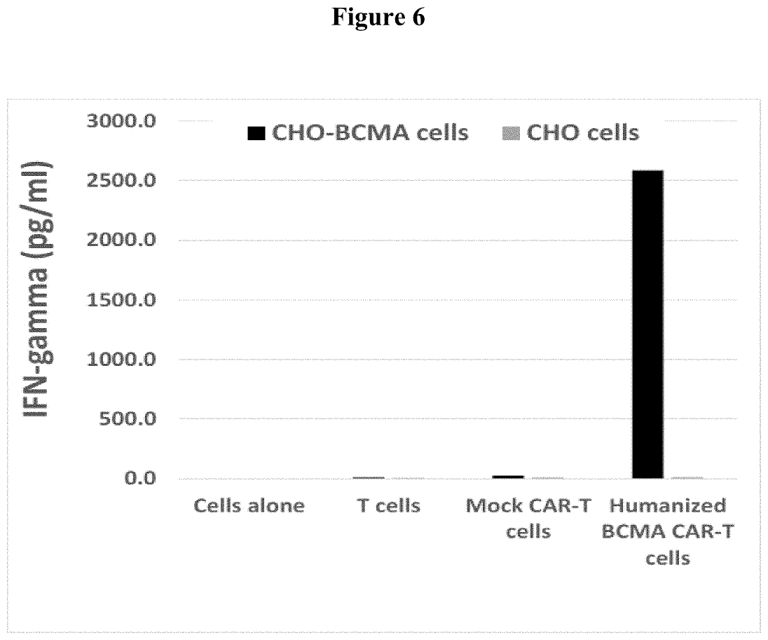

We collected supernatant after co-incubation of humanized BCMA-CAR-T cells and target CHO-BCMA and CHO cells and performed IFN-gamma assay. BCMA-CAR-T cells secreted IFN-gamma with CHO-BCMA cells but not with negative control CHO cells (FIG. 6). This confirms specificity of humanized BCMA-CAR-T cells and killing cytotoxicity assay.

FIG. 6 illustrates that humanized BCMA-CAR-T cells secreted high level of IFN-gamma with CHO-BCMA-positive cells but not with BCMA-negative CHO control cells. p<0.05 IFN-gamma in CHO-BCMA cells versus T and Mock CAR-T cells; and versus CHO cells.

### Example 6

### Humanized CAR-T Cells Secreted High Levels of IFN-Gamma Against BCMA-Positive RPMI8226 Multiple Myeloma Cells but not Against BCMA-Negative K562 Leukemia Cells

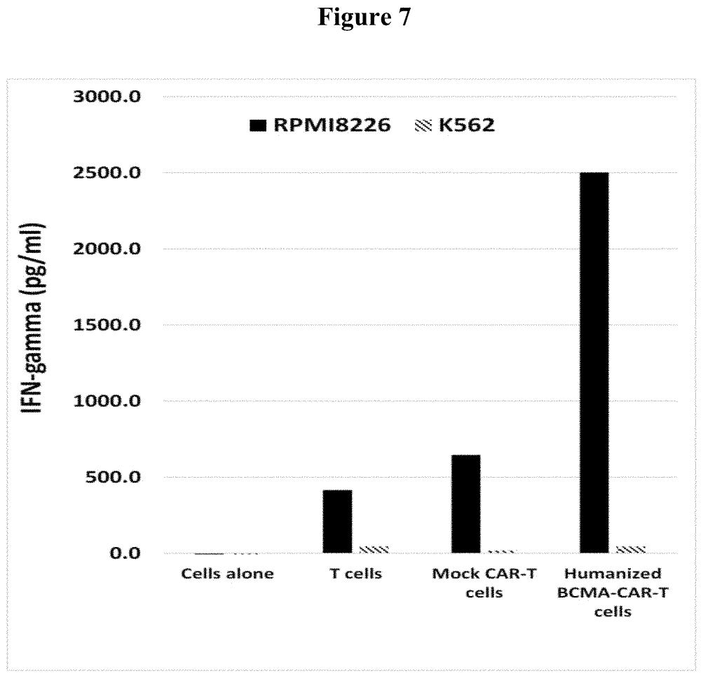

We incubated BCMA-CAR-T cells with multiple myeloma cancer cells RPMI8266, and BCMA-negative K562 cells and performed ELISA with IFN-gamma using kit from Fisher, according to their protocol. Humanized BCMA-CAR-T cells secreted high level of IFN-gamma against BCMA-positive multiple myeloma cancer cells but not against BCMA-negative K562 cells (FIG. 7). The level of killing and secretion of IFN-gamma was significantly higher than with T and Mock CAR-T cells. This confirms specificity of humanized BCMA-CAR-T cells against hematological BCMA-positive cells. We also compared humanized BCMA with mouse BCMA-CAR cells andwith humanized BCMA-CAR-T cells from other souce (used in clinical trials) and found similar or comparable or even higher secretion of IFN-gamma of our humanized BCMA-CAR-T cells with both positive control CAR-T (mouse and other humanized BCMA-CAR-T cells) (not shown). This supports high potential of humanized BCMA-CAR-T cells for future testing in clinic.

FIG. 7 shows that humanized BCMA-CAR-T cells secreted high level of IFN-gamma against multiple myeloma cells but not against BCMA-negative K562 control cells. p<0.05, IFN-gamma in multiple myeloma cells versus T and Mock-CAR-T cells; and versus IFN-gamma levels in K562 cells.

### Example 7

Humanized BCMA-CAR-T Cells Significantly Decreased RPMI8226 Xenograft Tumor Growth in Mouse Model In Vivo

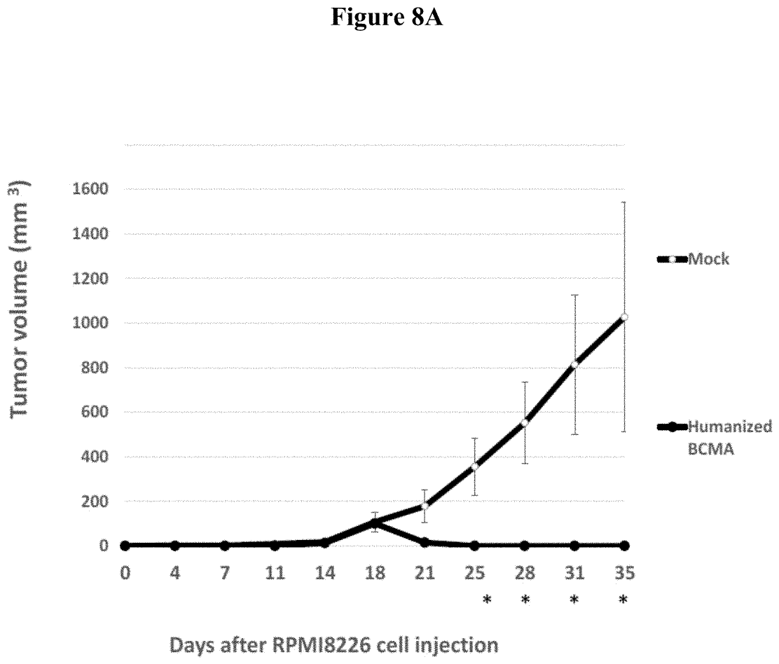

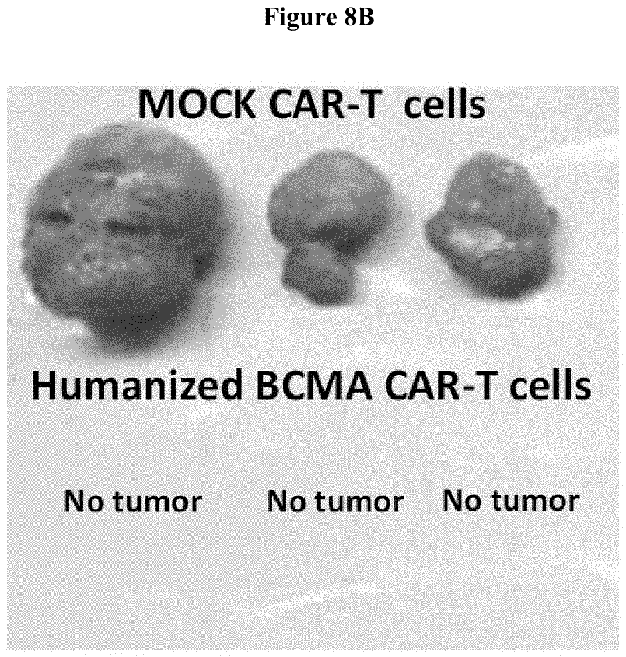

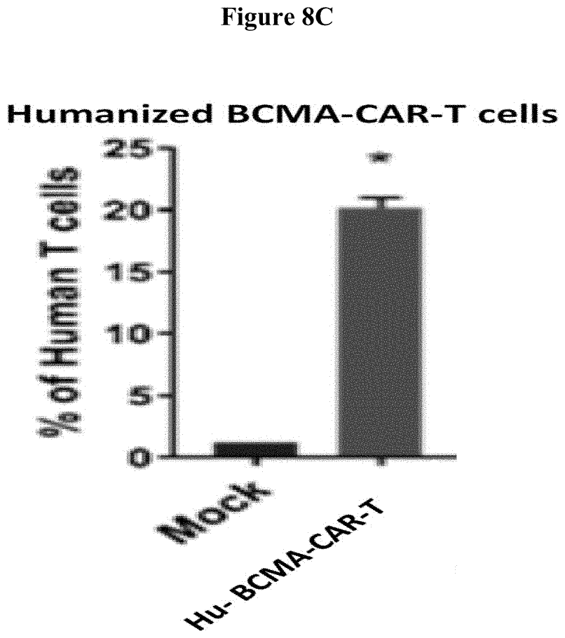

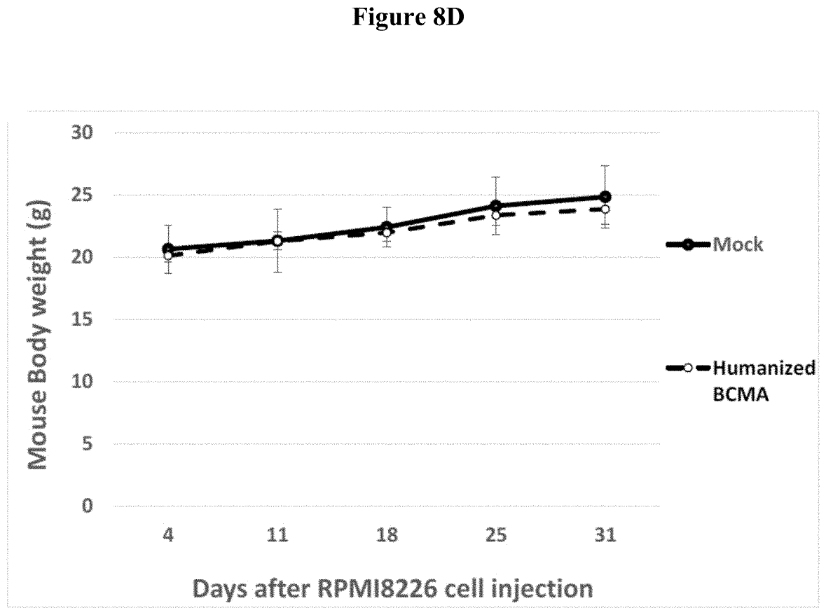

Multiple myeloma RPMI8226 cells were injected subcutaneously into NSG mice (1×10̂7 cells/mice), and then humanized BCMA-CAR-T cells were injected twice by i.v. (1×10̂7 CAR-T cells/mice). Humanized BCMA-CAR-T cells significantly decreased RPMI8226 tumor growth in mice (FIG. 8A). The representative images of mice xenograft tumors treated with BCMA-CAR-T cells (FIG. 8B). BCMA-CAR-T cells were detected in the mouse blood by FACS with BCMA recombinant protein (FIG. 8C) The mice treated with humanized BCMA-CAR-T cells did not cause decreased mice body weight suggesting that CAR-T cells were not toxic to mice (FIG. 8D). No behavior or visual changes were observed during the study.

FIGS. 8A to 8D illustrate that humanized BCMA-CAR-T cells significantly decreased RPMI8226 xenograft tumor growth. A. Humanized BCMA-CAR-T cells significantly decreased RPMI8226 tumor volume. CAR-T cells were injected at day 7 and 20 by i.v 1×10̂7 cells/mice. Bars show average tumor volume+/−standard errors. *p<0.05, BCMA vs Mock. B. Representative images of tumors treated with Mock control and Humanized BCMA-CAR-T cells. C. BCMA-CAR-T cells are detected in BCMA-CAR-T cell-treated mice by FACS with fluorescently-labelled BCMA recombinant protein. D. Humanized BCMA-CAR-T cells did not decreased mouse body weight. Bars show average mice body weight+/−standard deviations.

### Example 8

### Construction of a Humanized BCMA CAR

Applicants humanized the BCMA clone 4C8A scFv using established methods and inserted the scFv into a lentiviral vector expressing 2nd generation CAR domains (FIG. 3A). Human HEK293FT cells were transfected with the humanized BCMA lentiviral vector and lentiviral packaging plasmids. Two and three days after transfection, the culture medium was collected, clarified of cellular debris, and centrifuged at high speed to pellet the lentivirus. The viral pellet was suspended in aqueous buffer, divided into aliquots and frozen.

### Example 9

### Characterization of Humanized BCMA CAR-T Cells In Vitro

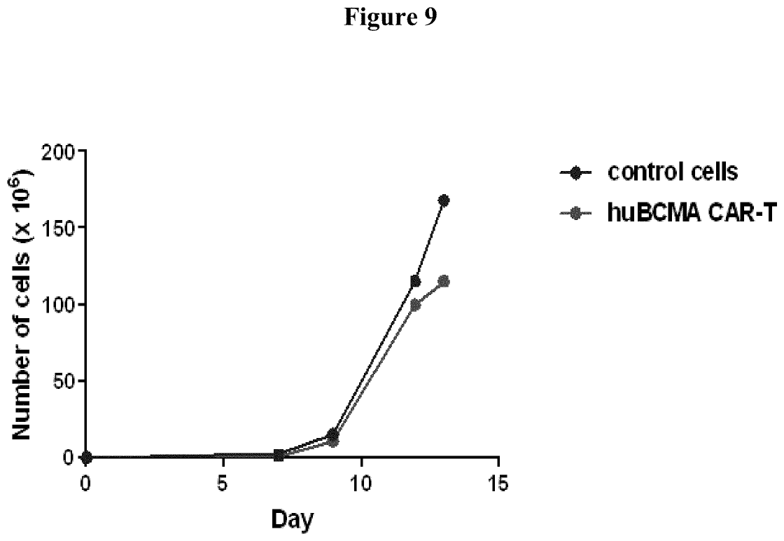

Human peripheral blood mononuclear cells (PBMC) were cultured overnight in AIM-V medium containing fetal bovine serum and human IL-2, along with Human T cell activator magnetic beads. The next day (day 1) an aliquot of huBCMA virus was added to the culture, along with DEAE-dextran to promote infection. On day 2 another aliquot of virus was added to the culture. The cells were cultured for another 11 days, during which additional medium was added to keep the cells at the correct density range. Over the 13-day culture period, the humanized BCMA CAR-T culture expanded approximately 250-fold (FIG. 9).

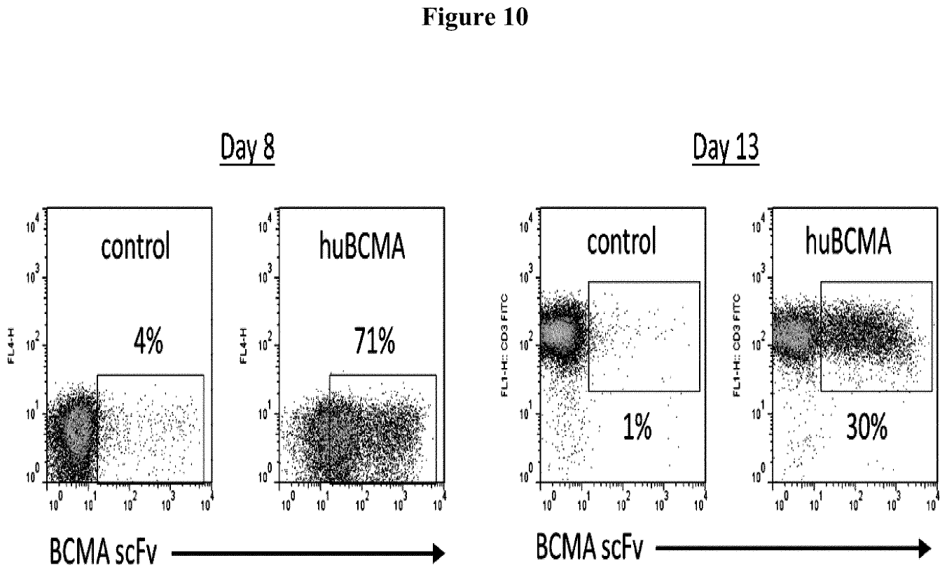

On days 8 and 13 the cells were analyzed by flow cytometry for the frequency of CAR-T cells. Cells were incubated with recombinant BCMA linked to a human IgG Fc fragment, and binding of the BCMA-Fc protein to the cells was detected with labeled goat anti-human IgG. Approximately 70% of the cells were CAR-T cells on day 8, which decreased to 30% on day 13 (FIG. 10).

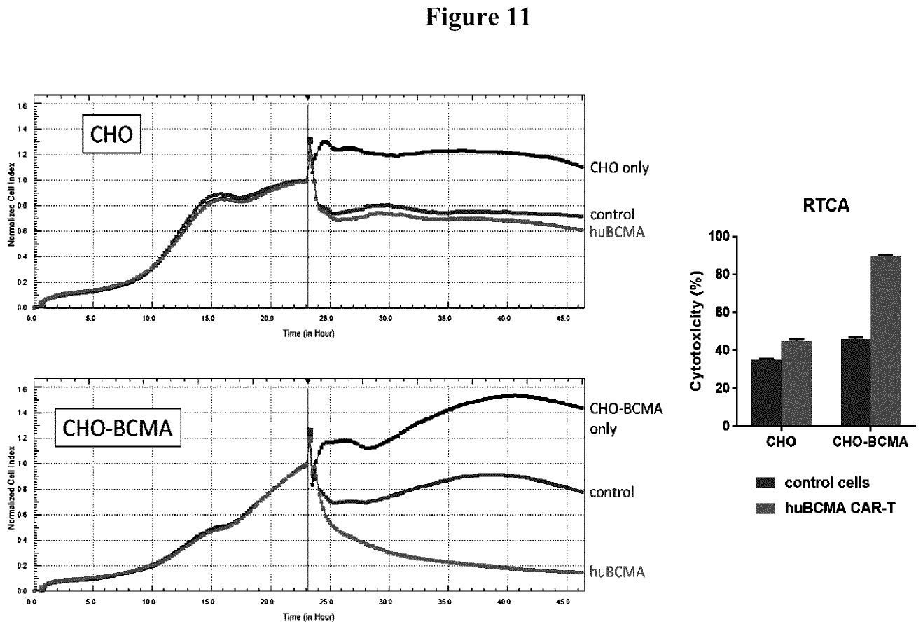

The huBCMA CAR-T cells were analyzed functionally in two ways. First, the cells were added to a monolayer of CHO cells stably expressing BCMA at an effector:target (E:T) cell ratio of 10:1, and cytolysis of the CHO-BCMA target cells was monitored by real-time cellular analysis (RTCA). Importantly, control T cells were added to the CHO-BCMA cells to indicate the extent of nonspecific cytolysis. In addition, the effector cells were added to a monolayer of CHO cells lacking BCMA. The huBCMA CAR-T cells killed almost 90% of the CHO-BCMA cells, whereas the control T cells killed only ˜40% of the CHO-BCMA cells (FIG. 11). In contrast, the huBCMA CAR-T cells and the control T cells both killed ˜40% of the CHO cells, indicating that the greater killing of CHO-BCMA cells by huBCMA CAR-T cells over control T cells is BCMA-dependent.

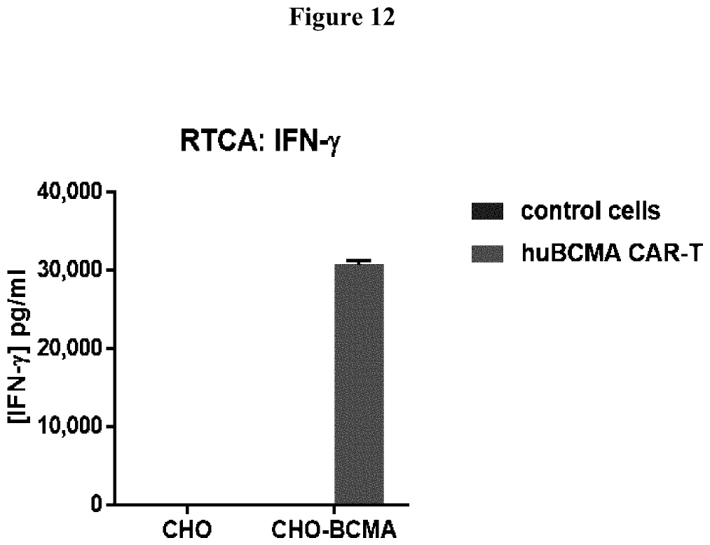

FIG. 12 is the real-time analysis of humanized BCMA CAR-T cell or control T cell cytolytic activity. CHO and CHO-BCMA cells were cultured for ˜23 hours, then huBCMA CAR-T cells or control T cells were added (vertical line). The Y-axis measures the impedance of the target cell monolayer, normalized to the time of CAR-T addition. Cytotoxicity was quantitated at the end of the assay with the formula (X−Y)*100/X, where X is the normalized impedance of the target cells alone and Y is the normalized impedance of the target cells plus the effector cells.

The humanized BCMA CAR-T cells were also analyzed for cytokine production. First, the culture medium from the RTCA assay was analyzed for IFN-γ, IL-2 and IL-6 levels. The huBCMA CAR-T cells produced high levels of IFN-γ but did not produce IL-2 and IL-6 (FIG. 12). The control T cells did not produce IFN-γ, indicating that the nonspecific killing of CHO and CHO-BCMA cells in the RTCA assay was IFN-γ-independent.

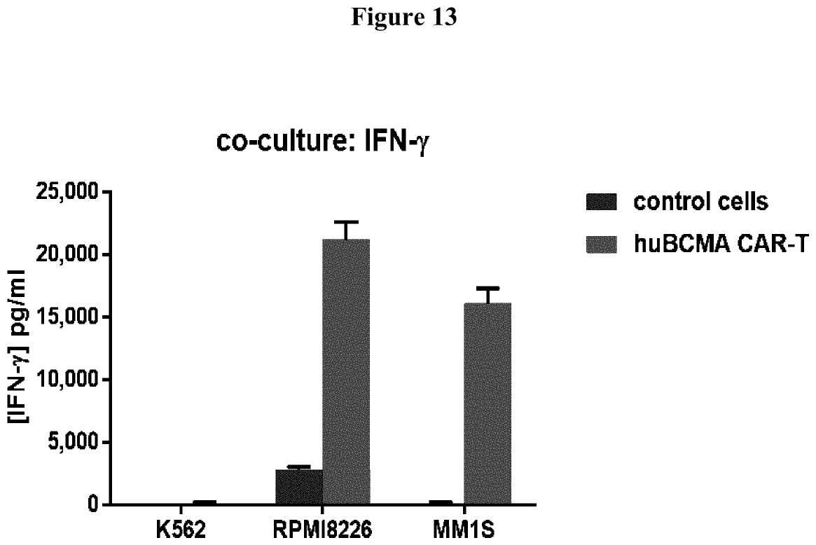

Cytokine production was also measured in a separate assay in which huBCMA CAR-T cells were cultured overnight with the BCMA-positive myeloma cell lines RPMI8226 and MM1S or the BCMA-negative cell line K562. Humanized BCMA CAR-T cells produced high levels of IFN-γ in response to each of the 2 myeloma cell lines but not in response to K562 cells (FIG. 13). As in the RTCA assay, huBCMA CAR-T cells did not produce IL-2 or IL-6.

In FIG. 13 there is described levels of IFN-γ produced by humanized BCMA CAR-T cells and control T cells during overnight culture with BCMA-positive myeloma cells RPMI8226 and MM1S or BCMA-negative K562 cells. Afterwards, the culture medium was centrifuged to remove cells and analyzed by ELISA.

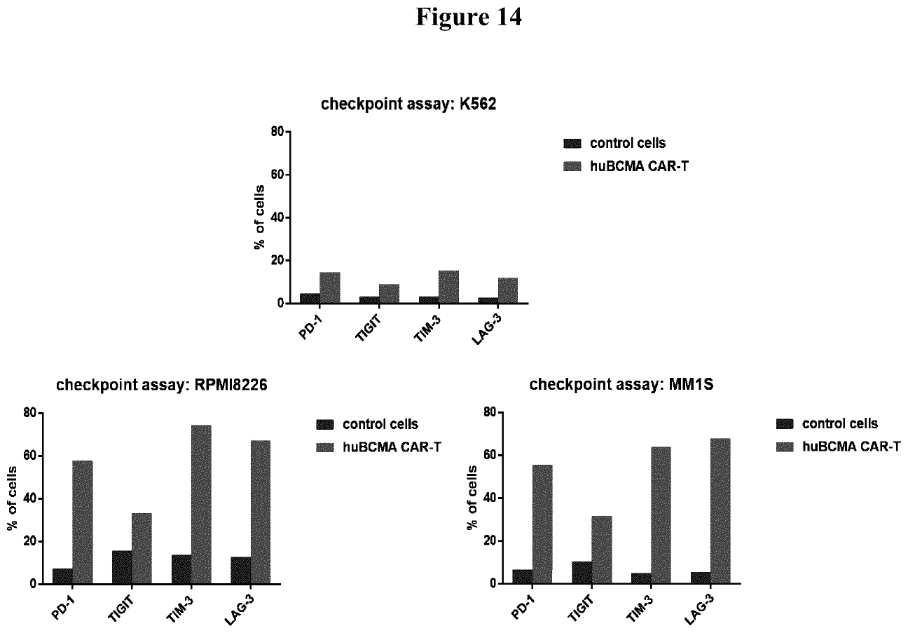

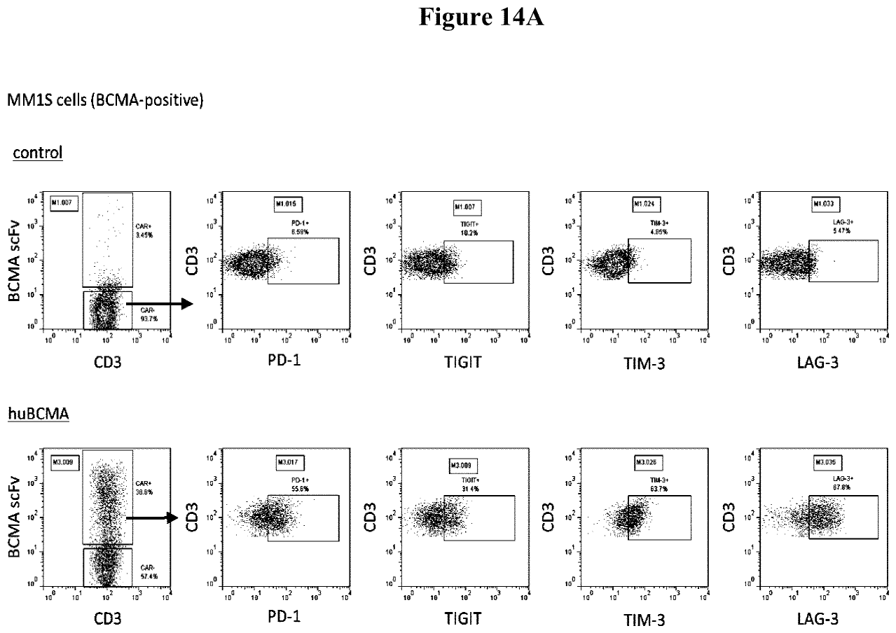

Lastly, huBCMA CAR-T cells were evaluated for checkpoint protein expression by flow cytometry after the overnight culture with myeloma cell lines. The BCMA-hFc protein was used to identify CAR-T cells, and the percentage of CAR-T cells expressing PD-1, TIGIT, TIM-3 or LAG-3 were measured. For the control T cells, the percentage of T cells expressing the checkpoint protein were measured. The flow cytometry plots are shown in FIG. 14A. After culture with BCMA-negative K562 cells, 9-15% of the huBCMA CAR-T cells expressed checkpoint proteins; in contrast, the percentages were 33-74% after culture with RPMI8226 cells and 31-68% after culture with MM1S cells (FIG. 14). Hence, checkpoint proteins were upregulated on huBCMA CAR-T cells after culture with target cells expressing BCMA.

FIG. 14 illustrates the percentages of humanized BCMA CAR-T cells or control T cells expressing PD-1, TIGIT, TIM-3 or LAG-3 after overnight culture with BCMA-positive myeloma cells RPMI8226 and MM1S or BCMA-negative K562 cells. The cells were analyzed by flow cytometry.

### Example 10

### Characterization of Humanized BCMA CAR-T Cells In Vitro: Multiple Donors

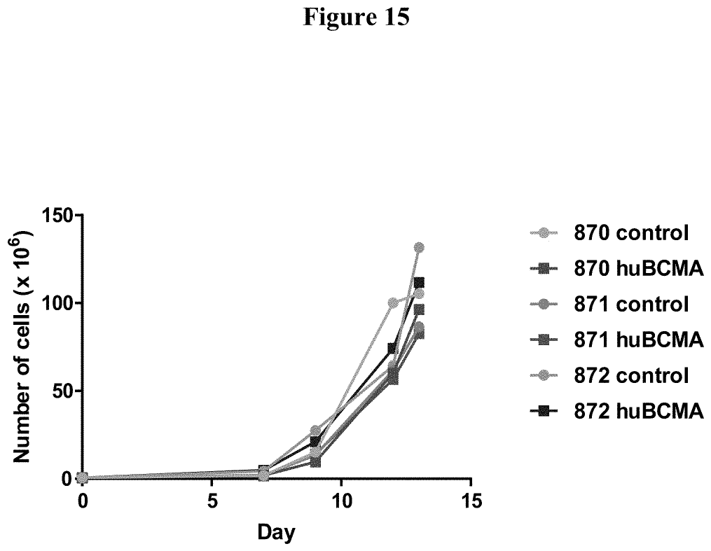

Human PBMC from 3 donors (#870, 871 and 872) were transduced with huBCMA lentivirus and CAR-T cells were cultured as described above. The expansion rates were comparable between the 3 donors and between the control T cells and the huBCMA CAR-T cells (FIG. 15).

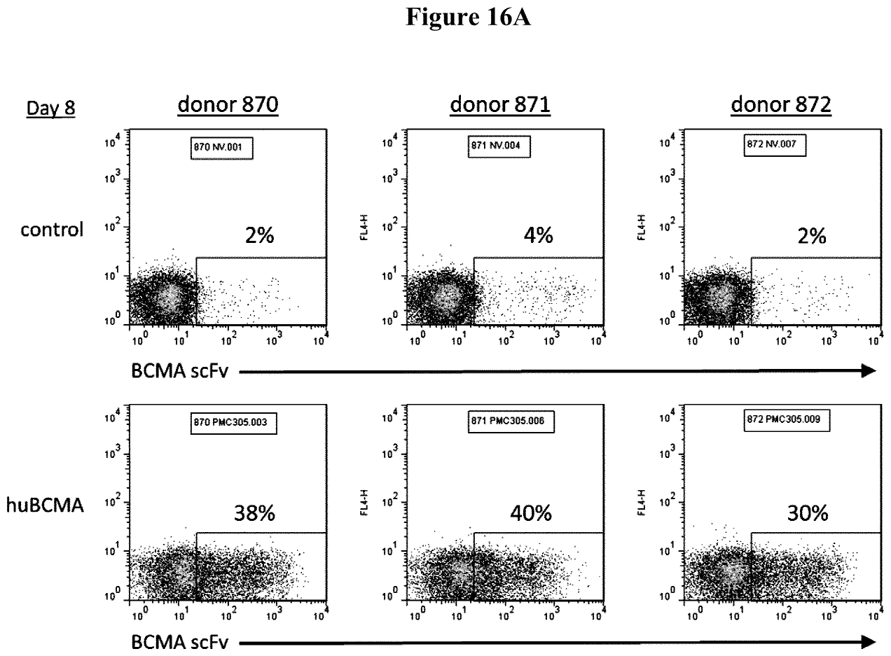

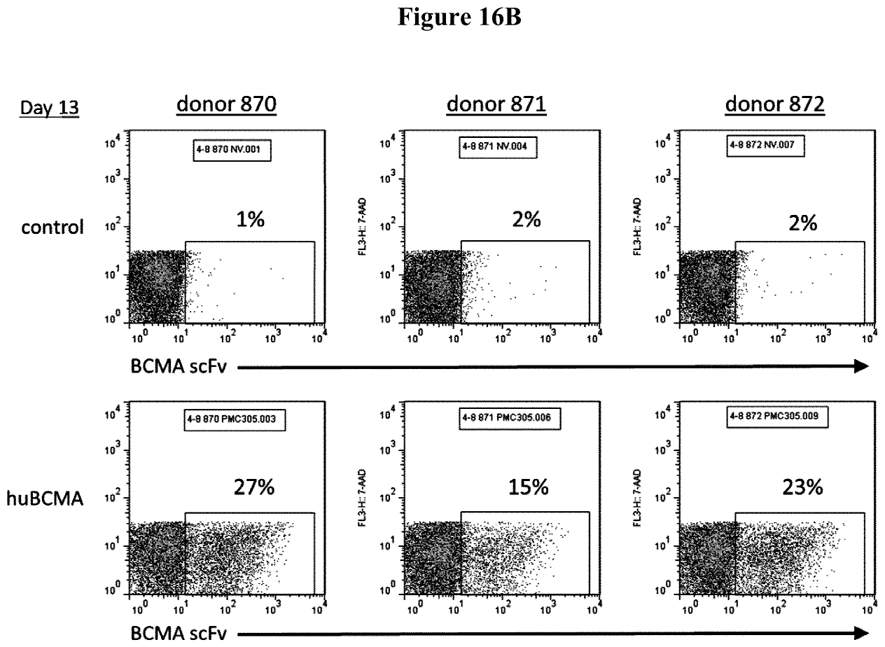

On day 8 of culture, 28-36% of the cells in each huBCMA culture were CAR-T cells, as determined by flow cytometry (FIGS. 16A and 16B). On day 13, the CAR-T frequency was 13-26%.

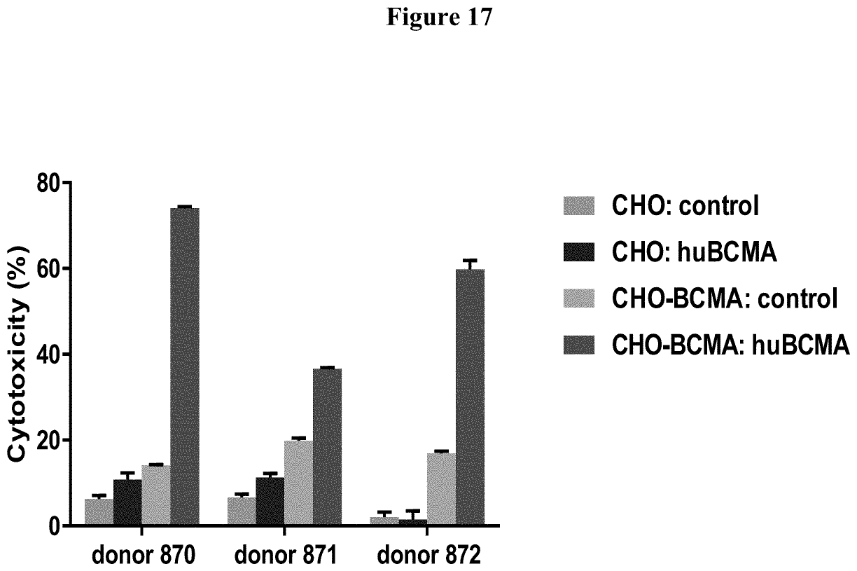

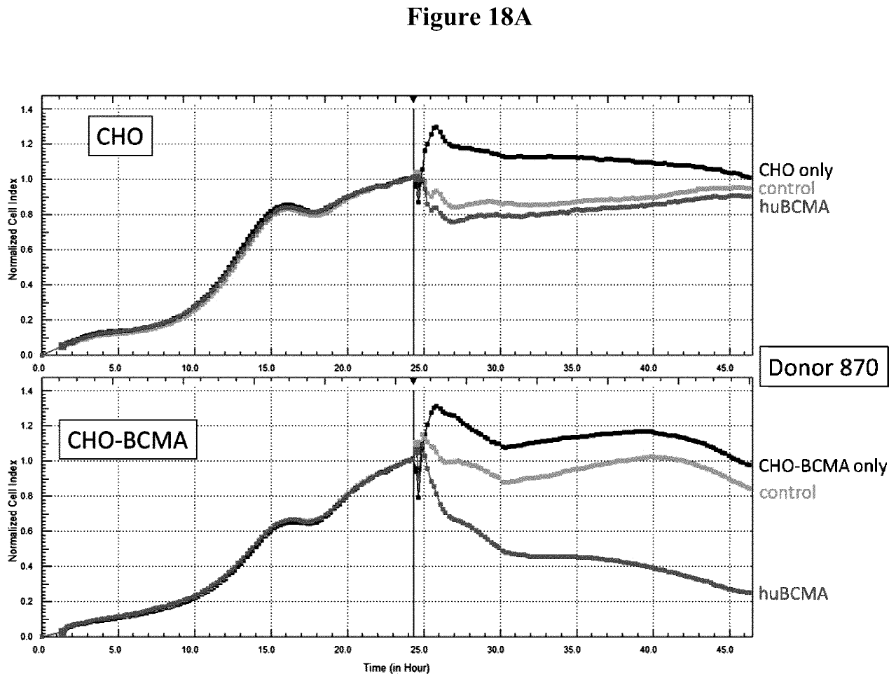

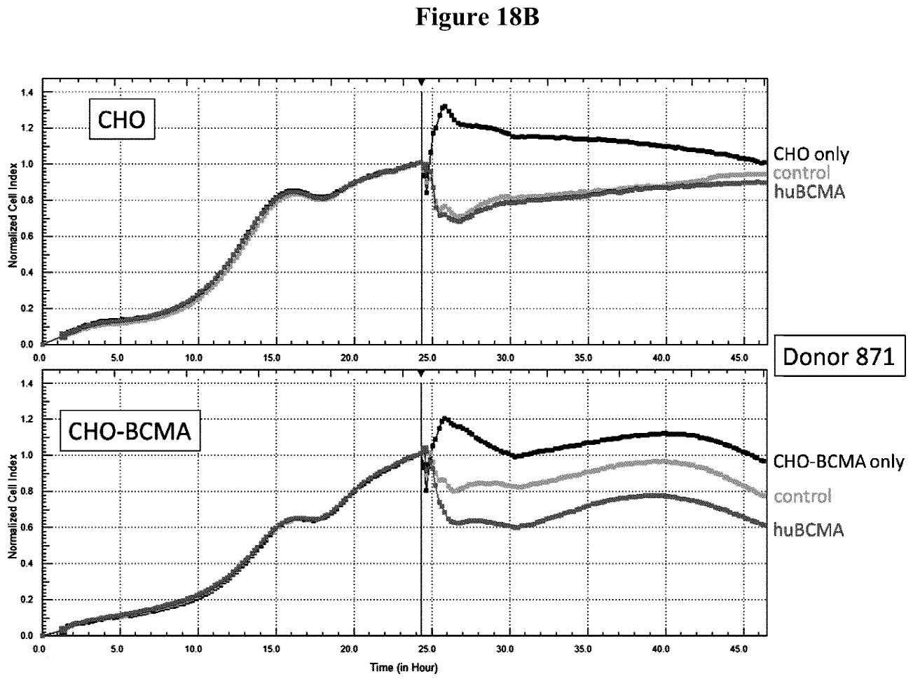

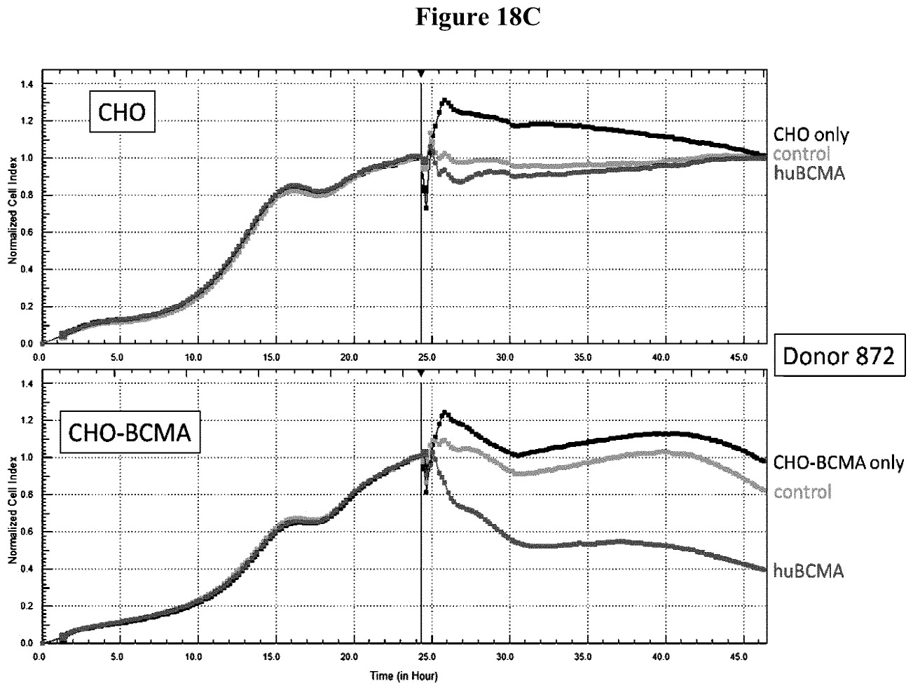

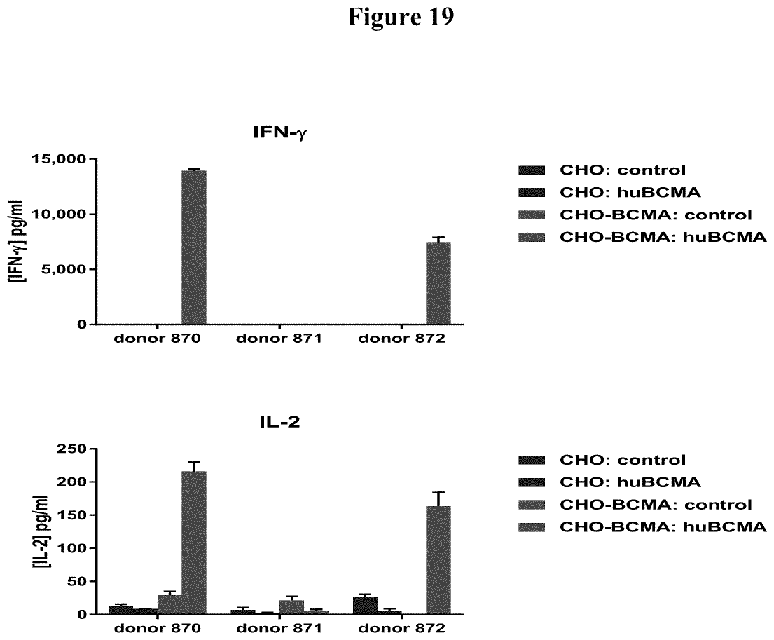

The CAR-T cells and control T cells were analyzed for cytotoxicity using RTCA with CHO and CHO-BCMA cells, as described above. The RTCA plots are shown in FIGS. 18A, 18B and 18C. HuBCMA CAR-T cells from donor 870 killed 74% of the CHO-BCMA cells; huBCMA CAR-T cells from donor 871 killed 36% of the CHO-BCMA cells; and huBCMA CAR-T cells from donor 872 killed 60% of the CHO-BCMA cells (FIG. 17). In contrast, huBCMA CAR-T cells from the 3 donors killed only 2-11% of the CHO cells, indicating that CAR-T cell killing of CHO-BCMA cells was largely BCMA-dependent. Control T cells killed 14-20% of the CHO-BCMA cells, indicating that some nonspecific killing occurred. The levels of IFN-γ and IL-2 in the RTCA culture medium were measured by ELISA. HuBCMA CAR-T cells from donors #870 and 872 produced high levels of IFN-γ and low levels of IL-2 in response to CHO-BCMA cells but not in response to CHO cells (FIG. 19). HuBCMA CAR-T cells from donor #871 did not produce IFN-γ or IL-2 in response to CHO-BCMA cells. None of the huBCMA CAR-T cultures produced IL-6 in response to CHO-BCMA cells.

### Example 11

### Characterization of Humanized BCMA CAR-T Cells In Vitro: T Cell Subsets

T cells are functionally divided into helper T cells, which are CD4+CD8− (“T4”), and cytotoxic T cells, which are CD8+CD4− (“T8”). T cells can also be divided based on differentiation status into 4 subsets: naïve (“Tn”), which are CD27+CD45RO−, central-memory (“Tcm”), which are CD27+CD45RO+, effector-memory (“Tem”), which are CD27−CD45RO+, and effector (“Teff”), which are CD27−CD45RO−. Tcm and Tem cells are the best choices for CAR-T therapy, since they are functionally active against cancer cells and are long-lived. Tn cells are long-lived but poorly active against cancer cells, whereas Teff cells are strongly active against cancer cells but are short-lived. Tn cells can differentiate into Tcm and Tem cells, so Tn cells are not bad for CAR-T therapy per se.

Human PBMC were separated into 4 subsets using antibodies and magnetic beads. The subsets were: CD4+CD8− T cells (T4), CD8+CD4− T cells (T8), CD4+central-memory T cells

(Tcm) and CD4+ effector-memory T cells (Tem). Immediately after isolation, the subsets and the PBMC were analyzed by flow cytometry for the composition of the subsets, using either CD4/CD8 antibodies or CD27/CD45RO antibodies.

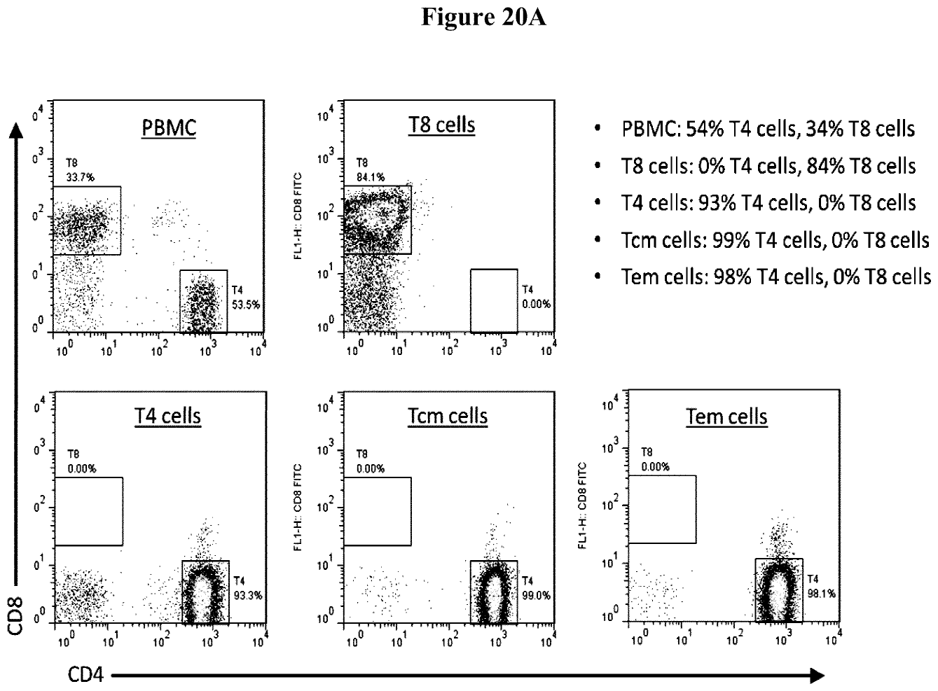

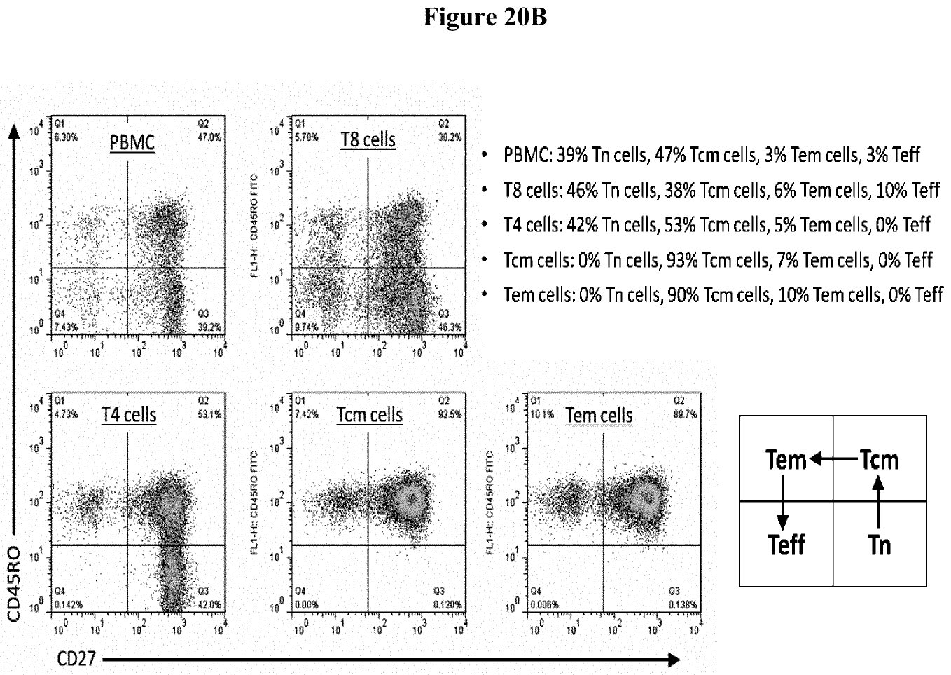

The T4, T8 and Tcm cells subsets well-purified: T4 cells were 93% T4, T8 cells were 84% T8, and Tem cells were 93% Tem (FIGS. 20A and 20B). However, the Tem subset contained mostly Tem cells, for reasons unknown.

FIGS. 20 and 20B illustrates the flow cytometric analysis of the PBMC and T cell subsets. The cells were first gated for live T cells using a CD3 antibody and 7-AAD (not shown). The cells were co-stained with antibodies against CD4 and CD8 (top 2 rows) or antibodies against CD27 and CD45RO (bottom 2 rows).

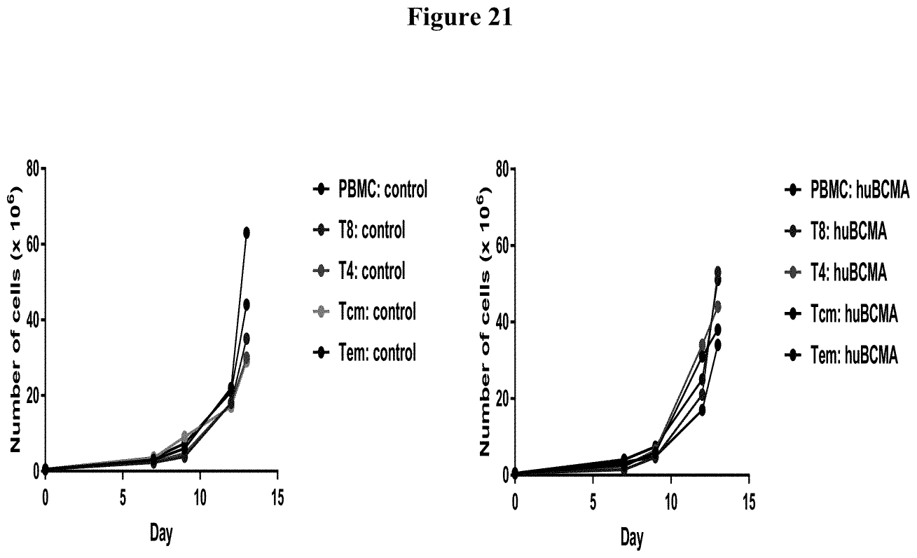

The 4 subsets and the PBMC were put into CAR-T culture, using Human T cell activator dynabeads. The next day (day 1) an aliquot of huBCMA virus was added to each culture, along with DEAE-dextran to promote infection. On day 2 another aliquot of virus was added to each culture. The cells were cultured for another 11 days, during which additional medium was added to keep the cells at the correct density range. Over the 13-day culture period, the cells expanded 60-120-fold (FIG. 21).

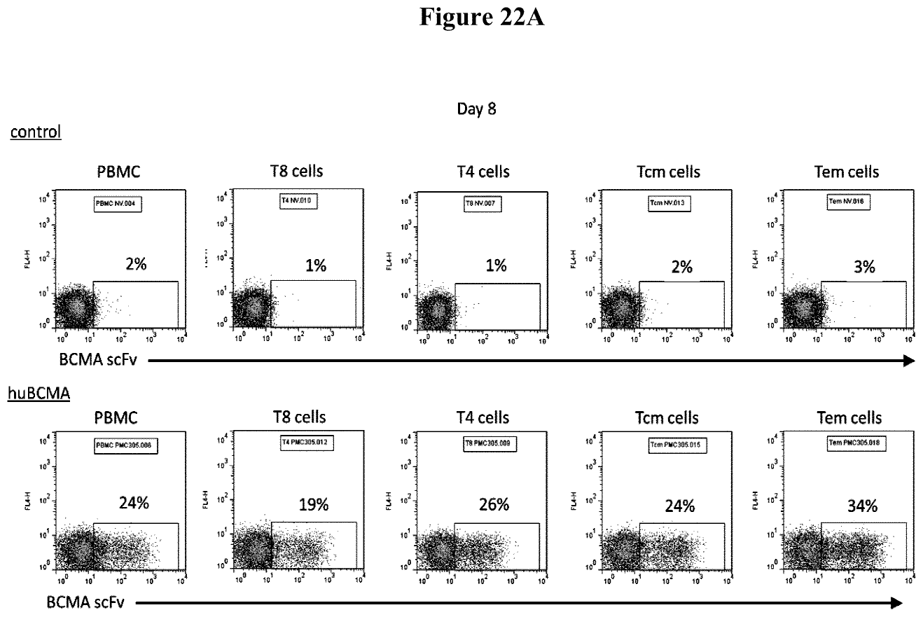

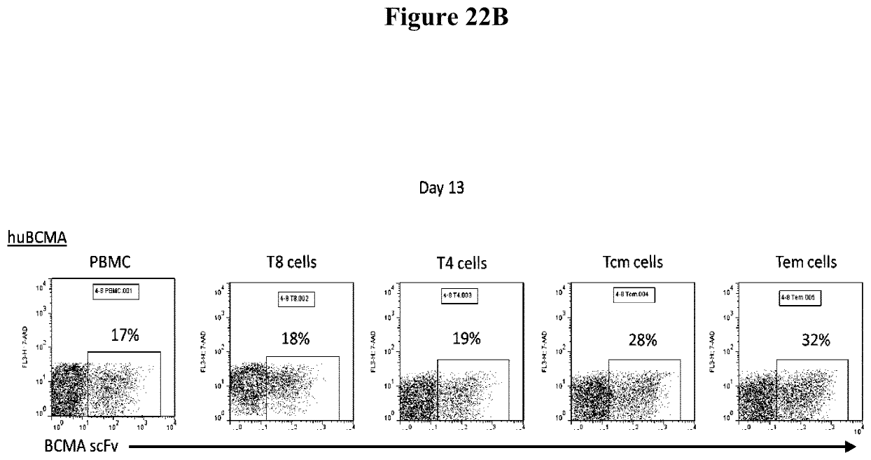

On days 8 and 13 the cells were analyzed by flow cytometry for the frequency of CAR-T cells. Cells were incubated with recombinant BCMA linked to a human IgG Fc fragment, and binding of the BCMA-Fc protein to the cells was detected with labeled goat anti-human IgG. Approximately 20-30% of the cells were CAR-T cells on day 8, and the percentage was decreased slightly on day 13 (FIGS. 22A and 22B).

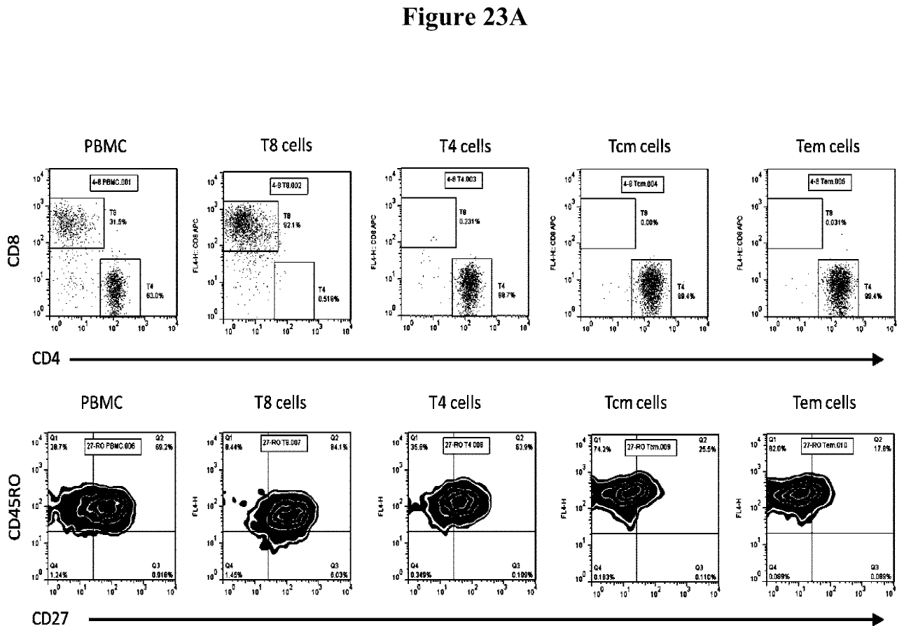

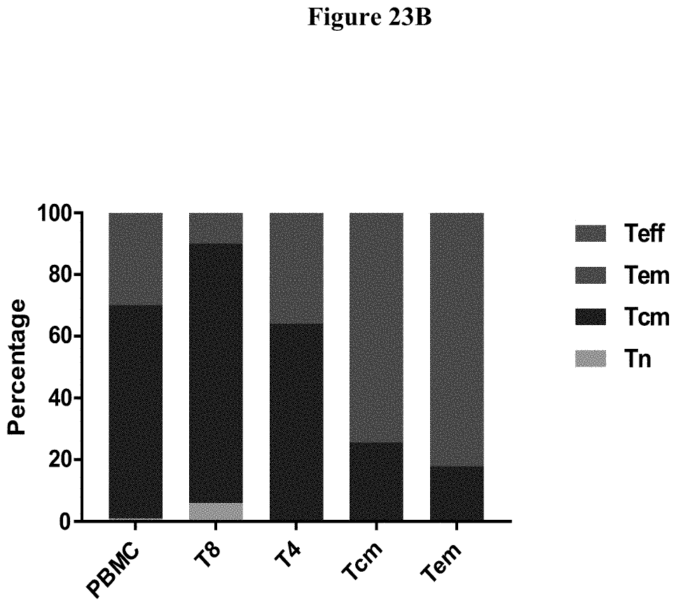

On day 13, the huBCMA CAR-T cells were analyzed by flow cytometry for CD4:CD8 ratios and differentiation subsets. Differentiation subsets were measured with antibodies for CD27 and CD45RO. The BCMA protein was included in the staining so that only the CAR-T cells were analyzed. CAR-T cells grown from the T4, Tem and Tem subsets were each entirely CD4+CD8−; CAR-T cells grown from the T8 subset were entirely CD8+CD4−, and CAR-T cells grown from PBMC were now a 2:1 mixture of T4 to T8 cells (FIGS. 23A and 23B). With regard to differentiation, CAR-T cells grown from the Tcm subset were now primarily Tem cells, similar to the Tem subset. CAR-T cells grown from PBMC, T4 and T8 subsets were primarily Tcm cells.

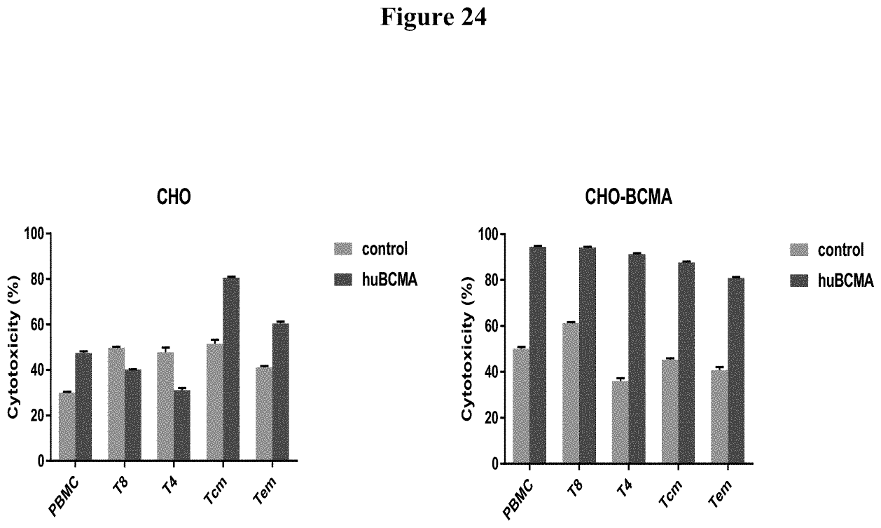

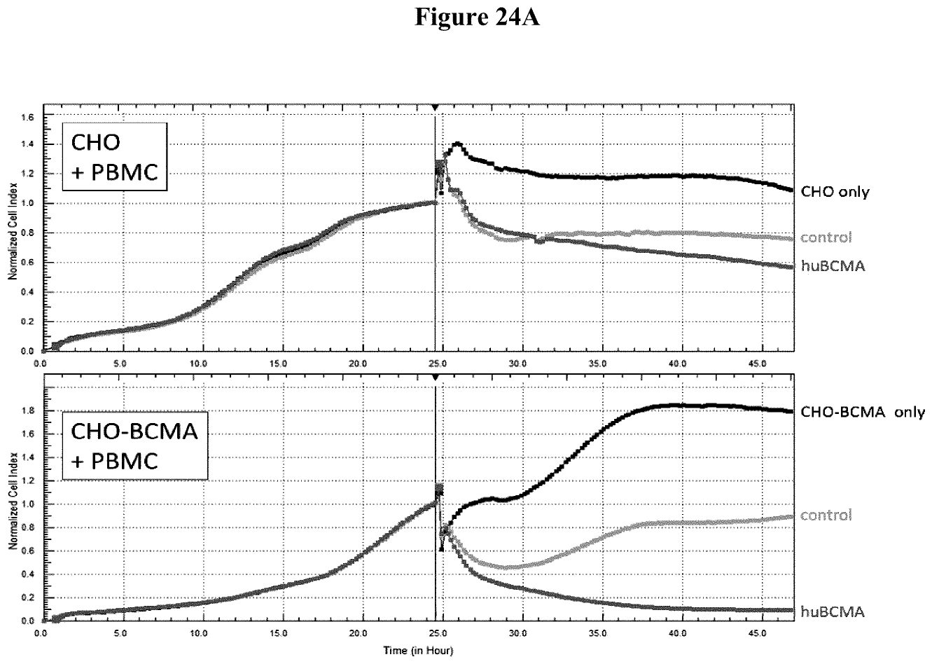

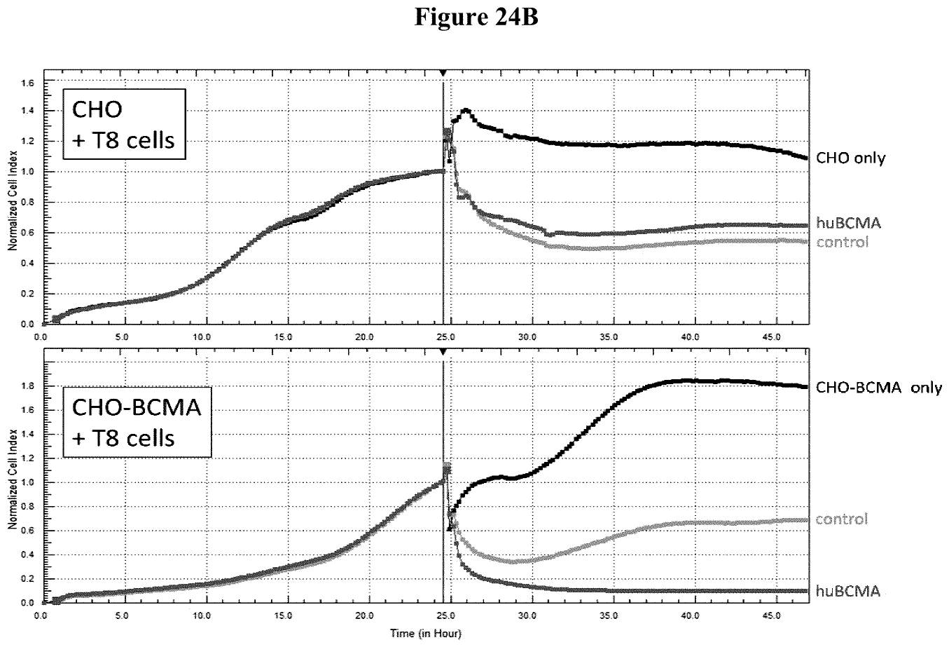

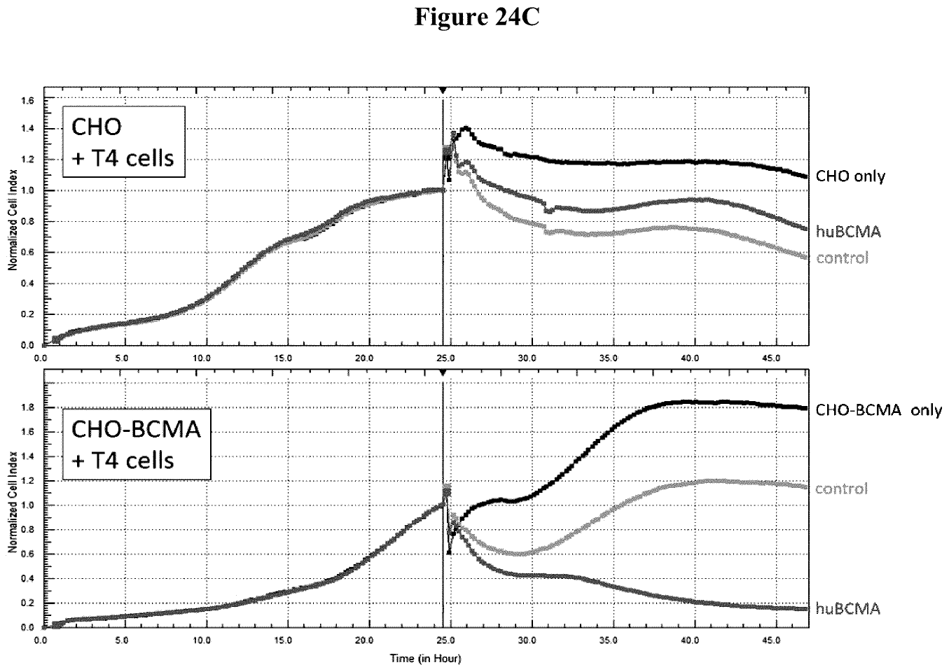

The day-13 CAR-T cells were tested for cytolytic activity on CHO and CHO-BCMA cells by RTCA assay. The RTCA plots are presented in FIGS. 24A, 24B and 24C. Although the levels of nonspecific killing were high (30-60%), for each T cell subset the huBCMA CAR-T cells were more cytotoxic than control T cells for CHO-BCMA target cells (FIG. 24).

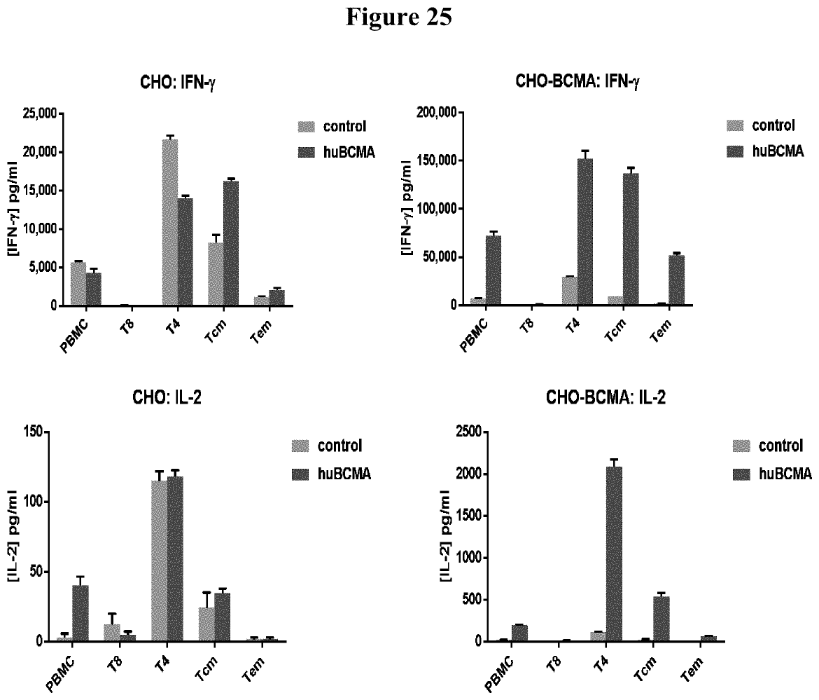

The levels of IFN-γ and IL-2 in the RTCA culture medium were measured by ELISA. Humanized BCMA CAR-T cells in all the cultures except the T8 culture produced high levels of IFN-γ in response to CHO-BCMA cells (FIG. 25). In response to CHO cells, moderate levels of IFN-γ were produced by both huBCMA CAR-T cells and control cells. The huBCMA CAR-T cells also produced IL-2 in response to CHO-BCMA cells; the T4 culture produced the most IL-2, followed by the Tcm and PBMC cultures, with little-to-no IL-2 produced by the T8 and Tem cultures. Very low levels of IL-2 were produced in response to CHO cells. FIG. 25 illustrates the levels of IFN-γ and IL-2 produced by huBCMA CAR-T cells or control T cells during the RTCA assay.

### Example 12

### Characterization of HuBCMA CAR-T Cells In Vitro: Manufacturing Medium

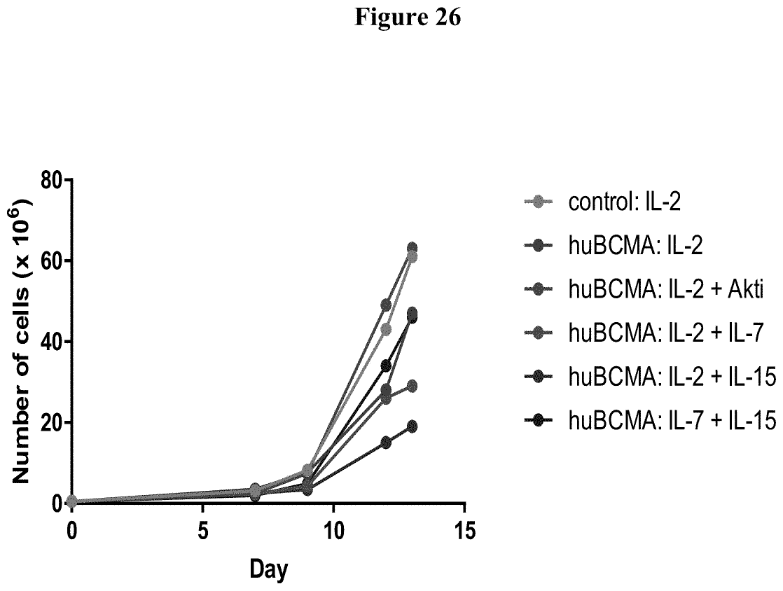

CAR-T cells are manufactured for clinical use by culturing in serum-free medium for 8-9 days. Before manufacturing huBCMA CAR-T cells, we first characterized their growth in serum-free medium containing different cytokines. Human PBMC were cultured overnight in AIM-V medium containing 5% Immune Cell Serum Replacement (Thermo Fisher) and either (1) 10 ng/ml IL-2, (2) 10 ng/ml IL-2 and 100 nM Atk inhibitor (Calbiochem # 124017), (3) 10 ng/ml IL-2 and 10 ng/ml IL-7, (4) 10 ng/ml IL-2 and 10 ng/ml IL-15, or (5) 10 ng/ml IL-7 and 10 ng/ml IL-15. T cells were activated with T cell activator magnetic beads, transduced with huBCMA virus, and CAR-T cells were cultured as described above. Over the 13-day culture period, the humanized BCMA CAR-T cultures expanded 40-120-fold (FIG. 26). FIG. 26 shows the expansion of humanized BCMA CAR-T cells over the culture period. The control culture was identical to the CAR-T culture but was not transduced with virus, and thus contains only human T cells.

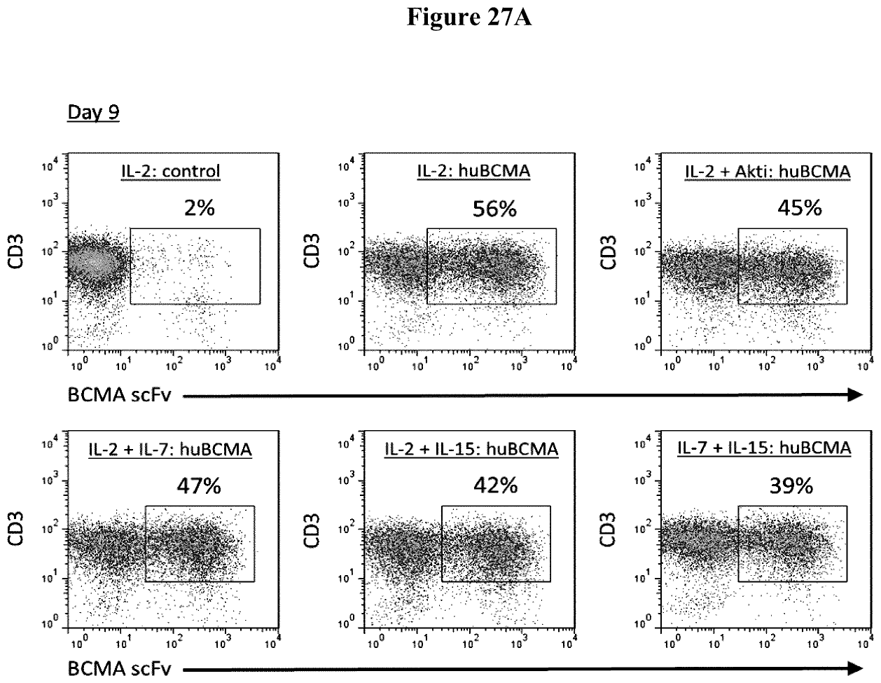

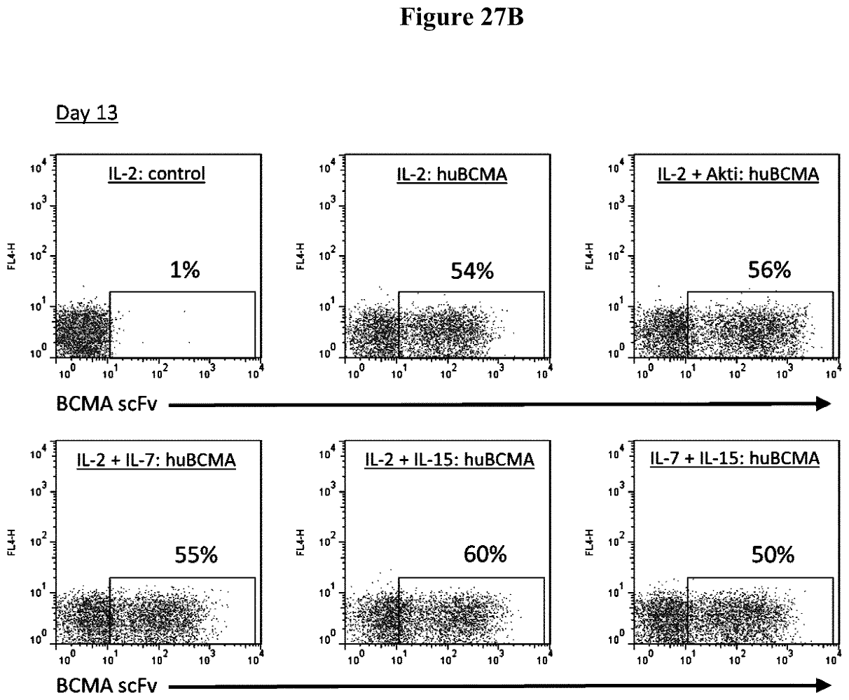

On days 9 and 13 the cells were analyzed by flow cytometry for the frequency of CAR-T cells. Cells were incubated with recombinant BCMA linked to a human IgG Fc fragment, and binding of the BCMA-Fc protein to the cells was detected with labeled goat anti-human IgG. Approximately 40-60% of the cells were CAR-T cells on both days 9 and day 13 (FIGS. 27A and 27B). FIGS. 27A and 27B show the flow cytometric analysis of BCMA protein binding to humanized BCMA CAR-T cells and control T cells on days 9 and 13 of culture. An anti-CD3 antibody was included on day 9 (Y-axis).

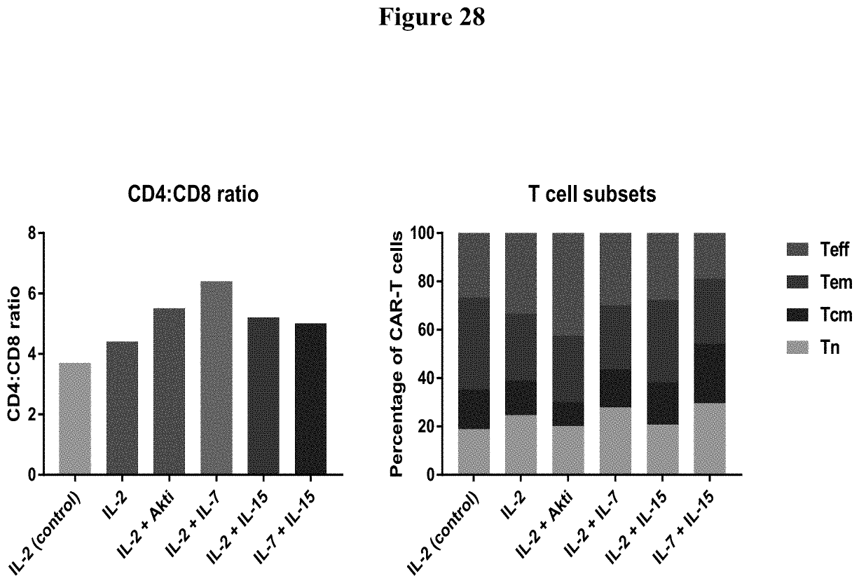

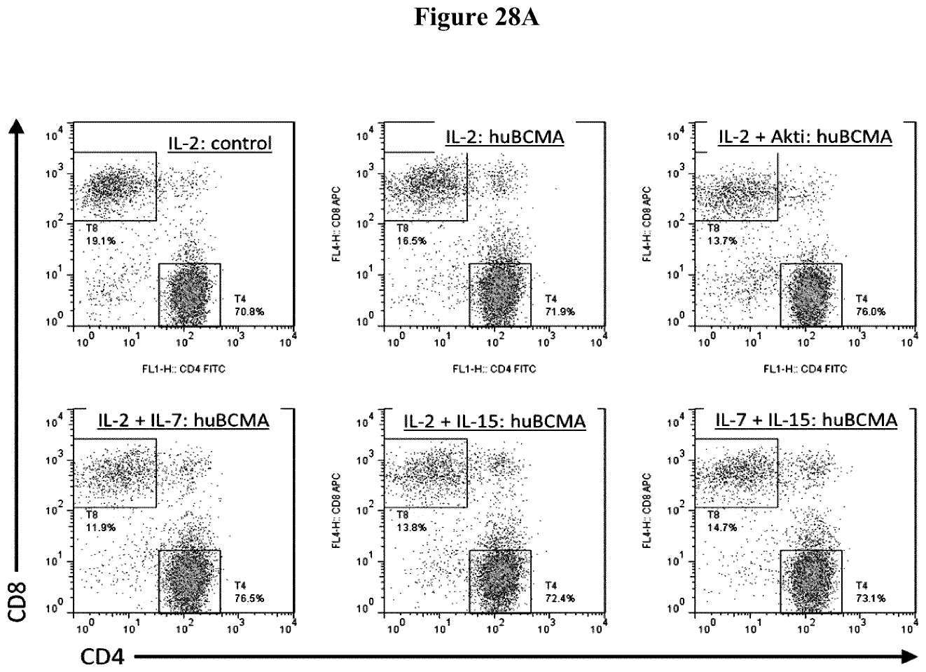

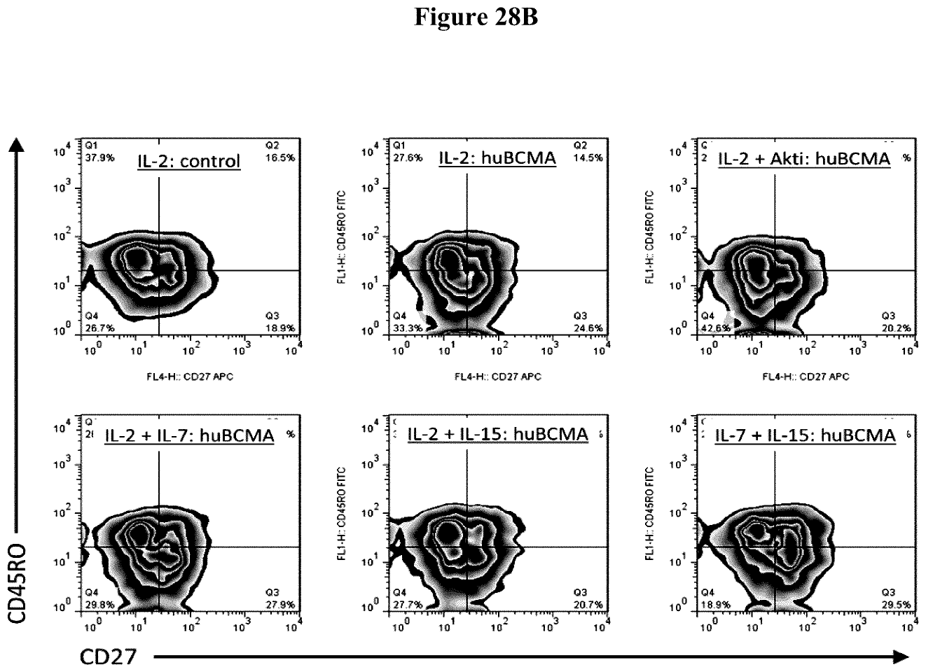

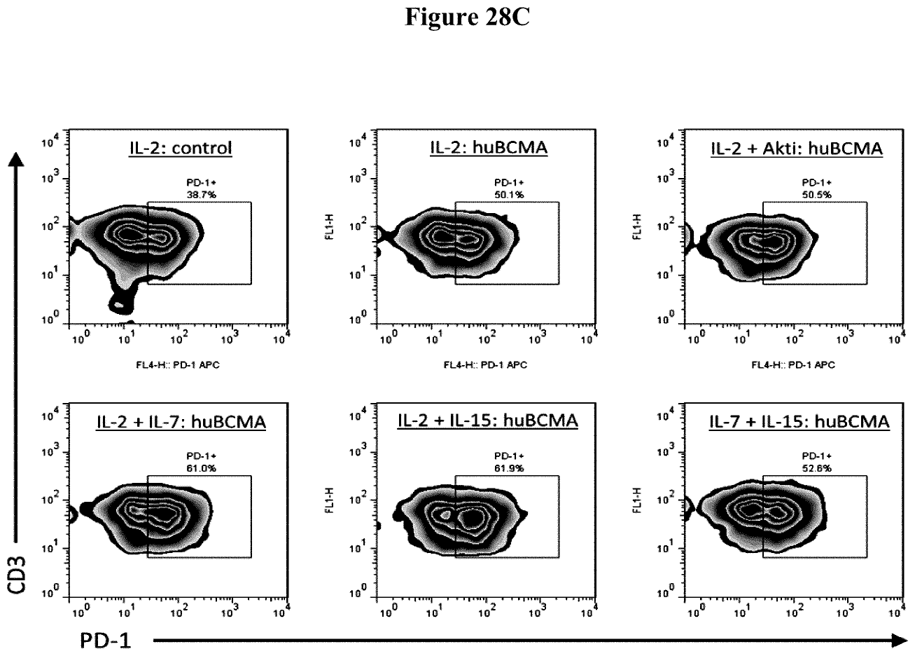

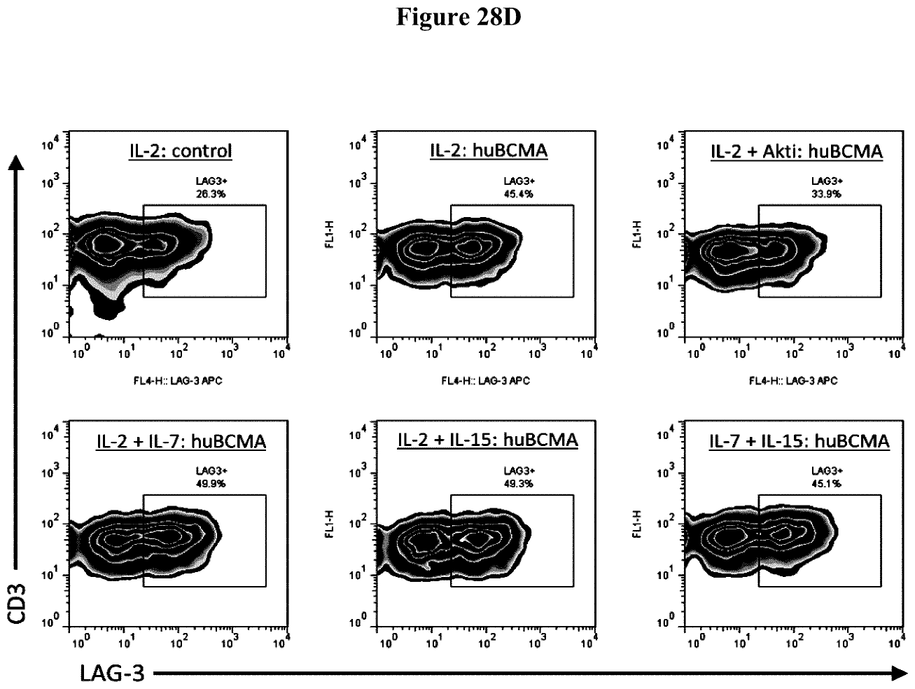

Since day 9 is often the day of harvest when CAR-T cells are manufactured, we analyzed the CAR-T cells on day 9 for T cell subsets, including T4 cells, T8 cells, Tn cells, Tcm cells, Tem cells and Teff cells. The FACS plots are shown in FIGS. 28A, 28B, 28C and 28D. HuBCMA CAR-T cells were predominantly CD4+in each of the cultures, with the highest frequency of T4 cells in the IL-2 +IL-7 culture (FIG. 28). The CAR-T cells in each of the cultures contained all 4 differentiation subsets. The CAR-T cells were most differentiated (highest percentage of Tem and Teff cells) in the IL-2+ Akti culture and least differentiated in the IL-7+ IL-15 culture.

FIG. 28 illustrates the flow cytometric analysis of huBCMA CAR-T cells. CAR-T cells were identified by BCMA protein binding and stained with antibodies for human CD4 and CD8 (left) or antibodies for human CD27 and CD45RO (right).

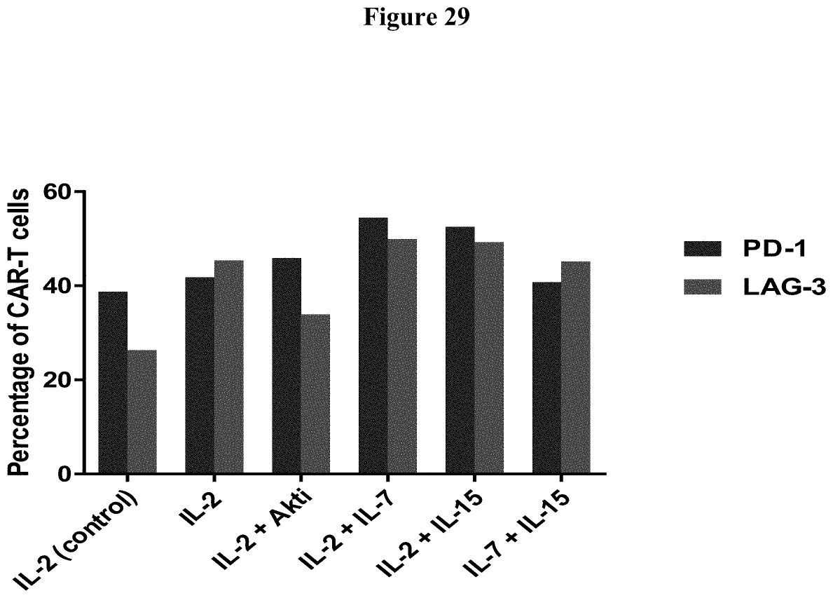

The huBCMA CAR-T cells were also analyzed for expression of checkpoint proteins PD-1 and LAG-3. The FACS plots are shown in Appendix 4. Approximately 30-50% of the CAR-T cells expressed the checkpoint proteins, with minor differences between the cultures (FIG. 29). FIG. 29 features the flow cytometric analysis of huBCMA CAR-T cells. CAR-T cells were identified by BCMA protein binding and stained with antibodies for human PD-1 or human LAG-3.

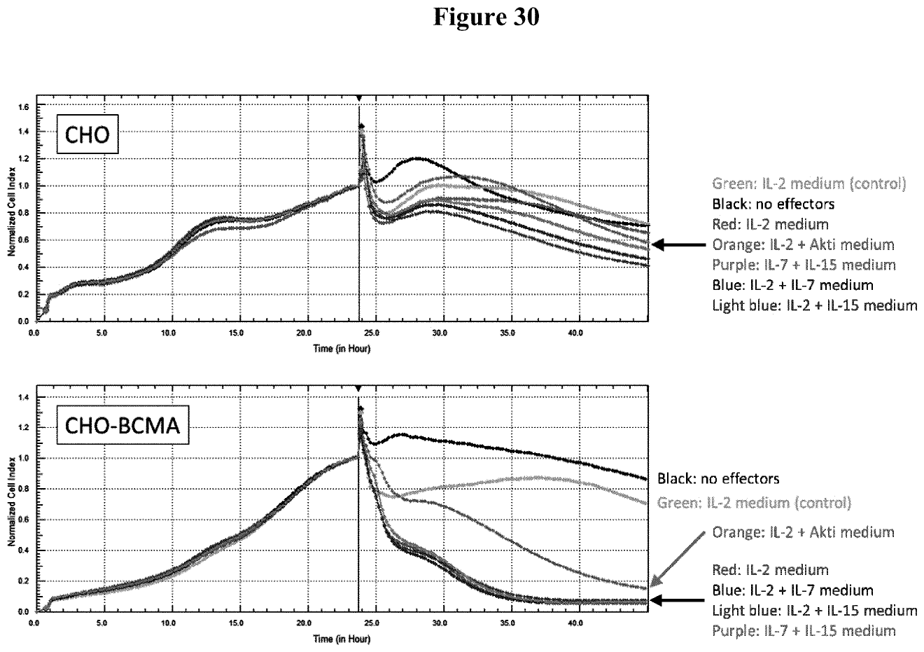

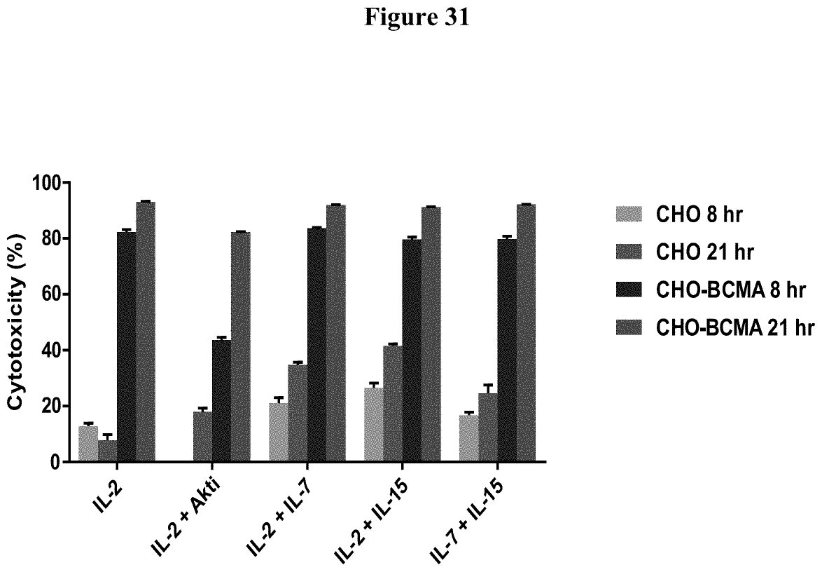

The huBCMA CAR-T cultures were analyzed functionally on day 13 by RTCA assay on CHO-BCMA cells and cytokine production during culture with myeloma cell lines. Most of the CAR-T cultures strongly killed CHO-BCMA cells, the exception being the cells in IL-2+ Akti, which killed CHO-BCMA cells with delayed kinetics (FIGS. 30-31). FIG. 30 is the real-time analysis of humanized BCMA CAR-T cell or control T cell cytolytic activity. CHO and CHO-BCMA cells were cultured for ˜24 hours, then the medium was removed and huBCMA CAR-T cells or control T cells were added in medium lacking cytokines (vertical line). The Y-axis measures the impedance of the target cell monolayer, normalized to the time of CAR-T addition. FIG. 31 shows the cytolytic activity of huBCMA CAR-T cells for CHO and CHO-BCMA target cells. The RTCA assay was performed as described above, with a 10:1 E:T ratio. Cytotoxicity was quantitated 8 and 21 hours after CAR-T cell addition.

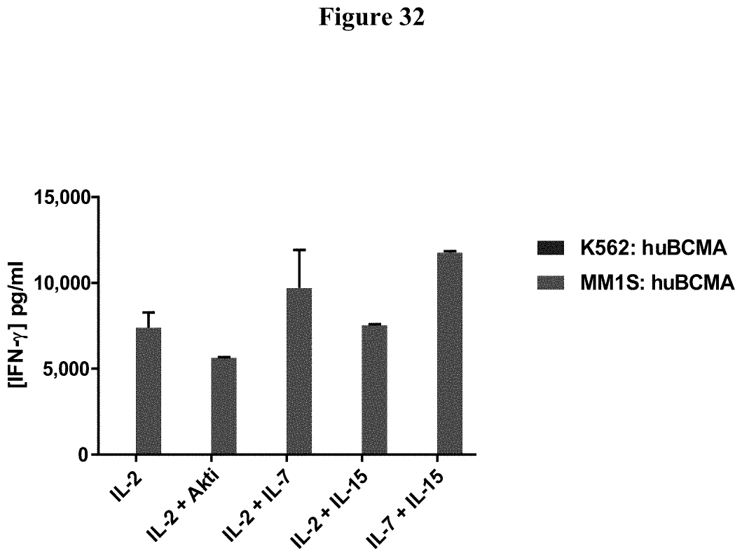

After overnight culture of huBCMA CAR-T cells with BCMA-negative K562 cells or BCMA-positive MM1S myeloma cells, the medium was analyzed for IFN-γ levels. As seen in prior experiments, huBCMA CAR-T cells produced IFN-γ in response to MM1S cells but not K562 cells (FIG. 32). The CAR-T cells cultured in IL-2 +Akti produced the least IFN-γ, whereas the CAR-T cells cultured in IL-7+ IL-15 produced the most IFN-γ. FIG. 32 describes the levels of IFN-γ produced by humanized BCMA CAR-T cells during overnight culture with BCMA-positive MM1S myeloma cells or BCMA-negative K562 cells. Afterwards, the culture medium was centrifuged to remove cells and analyzed by ELISA.

### Example 13

### Manufacturing and Testing of Humanized BCMA CAR-T Cells

Applicants manufactured huBCMA CAR-T cells over 9 days as follows. Thirty million human PBMC were cultured overnight with 30 million T cell activator magnetic beads in 30 ml of AIM-V medium containing 5% Immune Cell Serum Replacement and 10 ng/ml IL-2. The next day (day 1) an aliquot of huBCMA virus was added to the culture, along with DEAE-dextran to promote infection. On day 2 another aliquot of virus was added to the culture, and on day 4 40 ml of fresh medium was added to the culture. On day 5 the cells were transferred to a 1-liter G-Rex container containing 530 ml of fresh medium (total volume 600 ml). On day 9 the cells were counted and assayed. There were 1.8 billion live cells, indicating that the cells expanded 60-fold over the 9-day culture period.

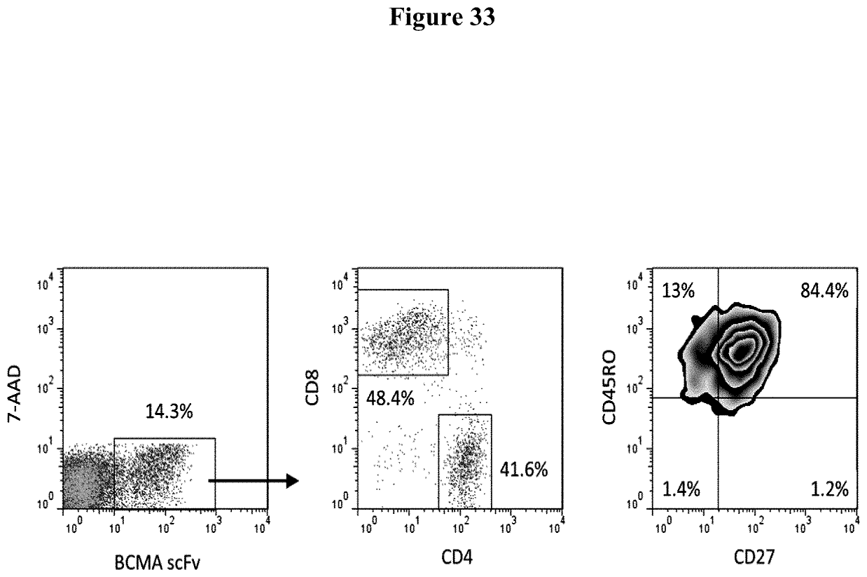

The cells were analyzed by flow cytometry for the frequency of CAR-T cells, the expression of CD4 and CD8 on the CAR-T cells, and the differentiation status of the CAR-T cells. Manufactured cells were incubated with recombinant BCMA linked to a human IgG Fc fragment, and binding of the BCMA-Fc protein to the cells was detected with labeled goat anti-human IgG. The cells were additionally incubated with labeled antibodies against CD4 and CD8 or CD27 and CD45RO. Fourteen percent of the cells were CAR-T cells, the CAR-T cell CD4:CD8 ratio was approximately 1:1, and the vast majority of CAR-T cells were Tcm cells (FIG. 33). FIG. 33 illustrates the flow cytometric analysis of manufactured huBCMA CAR-T cells. Cells were first plotted for binding to BCMA protein vs 7-AAD staining, and live CAR-T cells were gated. The gated cells were then plotted for CD4 vs CD8 staining and CD27 vs CD45RO staining.

The manufactured huBCMA CAR-T cells were analyzed in the RTCA assay with CHO-BCMA cells. Control T cells cultured in a 1-liter G-Rex container were used as negative control effector cells, and CHO cells were used as negative control target cells. Unfortunately, both the control T cells and the huBCMA CAR-T cells were cytotoxic for both the CHO and CHO-BCMA target cells, indicating that this particular PBMC donor was highly reactive for CHO cells (see Appendix 5).

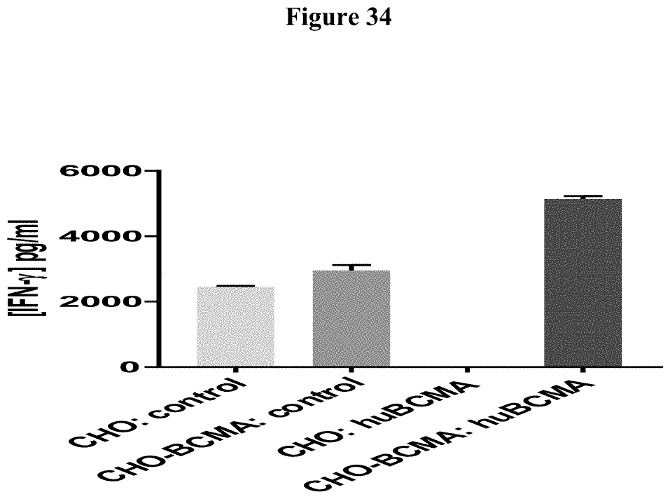

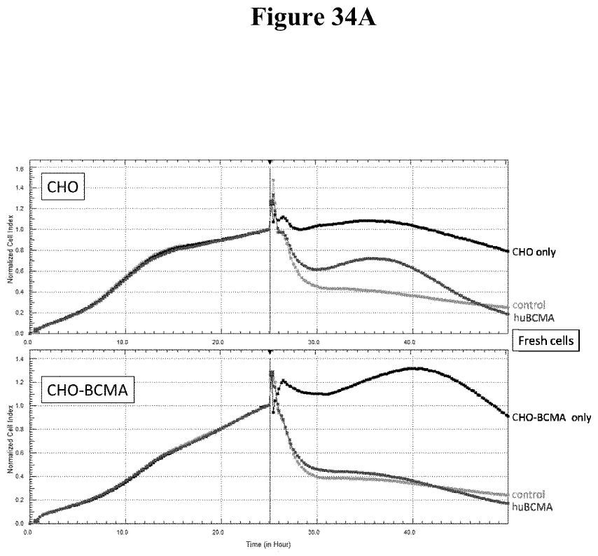

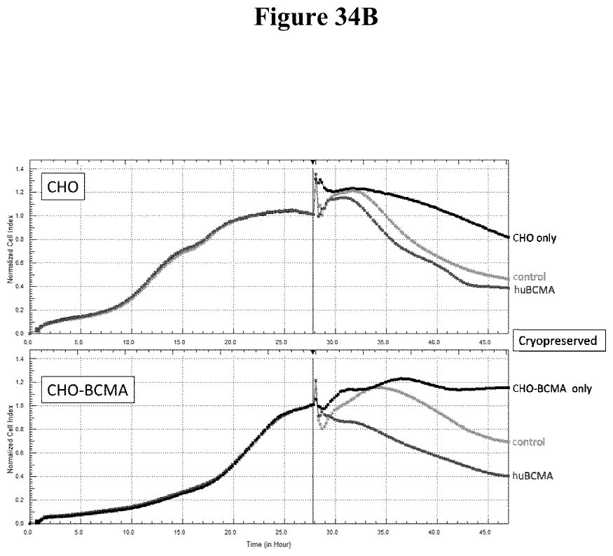

Since cytokine production occurs upon antigen recognition and signaling though the CAR's CD3□ chain, the levels of IFN-γ and IL-2 in the RTCA culture medium were measured by ELISA. As expected, the manufactured huBCMA CAR-T cells produced IFN-γ only in response to CHO-BCMA cells, not CHO cells (FIG. 34). The control T cells also produced IFN-γ, but in response to both CHO and CHO-BCMA cells. Neither control T cells nor manufactured huBCMA CAR-T cells produced IL-2. FIG. 34 illustrates the levels of IFN-γ produced by manufactured huBCMA CAR-T cells and control T cells during the RTCA assay.

The manufactured huBCMA CAR-T cells were analyzed for the presence of endotoxin, mycoplasma and bacteria. Endotoxin levels were below the level of detection (0.1 EU/ml) in a chromogenic amebocyte lysate assay (Thermo Fisher # A39552). Mycoplasma was also undetectable, by PCR (Sigma # MP0035). Bacteria could not be cultured from the CAR-T cells on Luria broth agar plates.

One-hundred fifty million manufactured huBCMA CAR-T cells were cryopreserved in a 250-ml cell bag using 30 ml of GMP-grade freezing medium. Freezing was accomplished using a controlled-rate freezer, and the frozen bag was stored in liquid nitrogen for 4 days. The cells were thawed by partial immersion of the bag in a 37° C. water bath, then cells were withdrawn from the bag and rinsed in manufacturing medium. Cell counting with trypan blue indicated that 54 million live cells and 19 million dead cells were recovered.

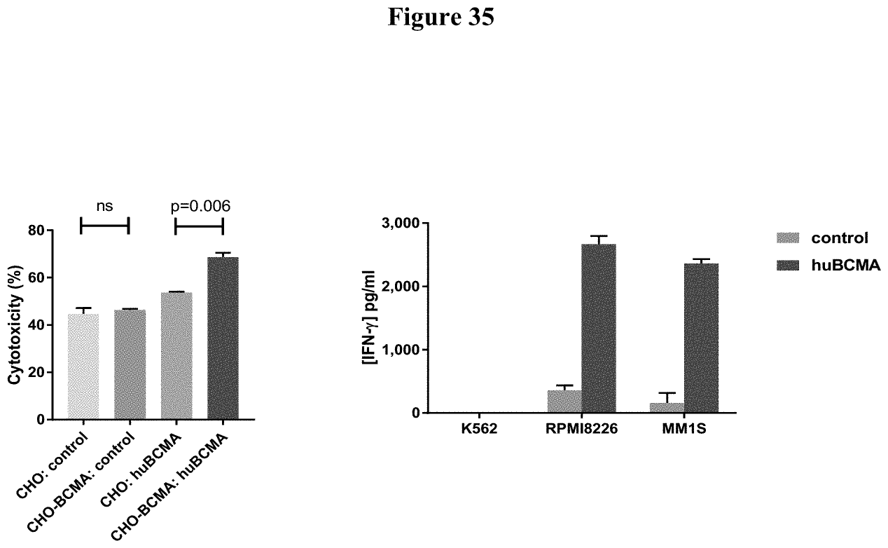

The thawed cells were analyzed by RTCA assay and by coculturing with multiple myeloma cells. Similar to the cells before freezing, the cryopreserved huBCMA CAR-T cells and control T cells were cytotoxic for CHO cells, but not as severe (FIGS. 34A and 34B). The decrease in the severity revealed the preferential cytotoxicity of huBCMA CAR-T cells for CHO-BCMA cells over CHO cells (FIG. 35). In contrast, control T cells were equally cytotoxic for CHO and CHO-BCMA cells. In the coculture, the freshly-thawed huBCMA CAR-T cells produced IFN-γ in response to BCMA+RPMI8226 and MM1S myeloma cells but not in response to BCMA-negative K562 cells (FIG. 35). FIG. 35 features the analysis of cryopreserved manufactured huBCMA CAR-T cells. Left: cytolytic activity of cryopreserved manufactured huBCMA CAR-T cells and control T cells for CHO and CHO-BCMA target cells. The RTCA assay was performed as described above, with a 10:1 E:T ratio. p values are shown above (1-way ANOVA with Tukey's post-hoc test). Right: levels of IFN-γ produced by cryopreserved manufactured huBCMA CAR-T cells and control T cells after overnight culture with BCMA-negative K562 cells and BCMA+RPMI8226 and MM1S myeloma cells.

A portion of the cryopreserved manufactured huBCMA CAR-T cells was put back into culture immediately after thawing, at 1 million live cells per ml of manufacturing medium containing IL-2. Two days later the cells were counted. The live cells were now at a density of 2.62 million per ml, indicating that the cryopreserved cells were healthy and capable of proliferating in culture.

### Example 14

### Characterization of Humanized BCMA CARr-T Cells in a Xenograft Tumor Model

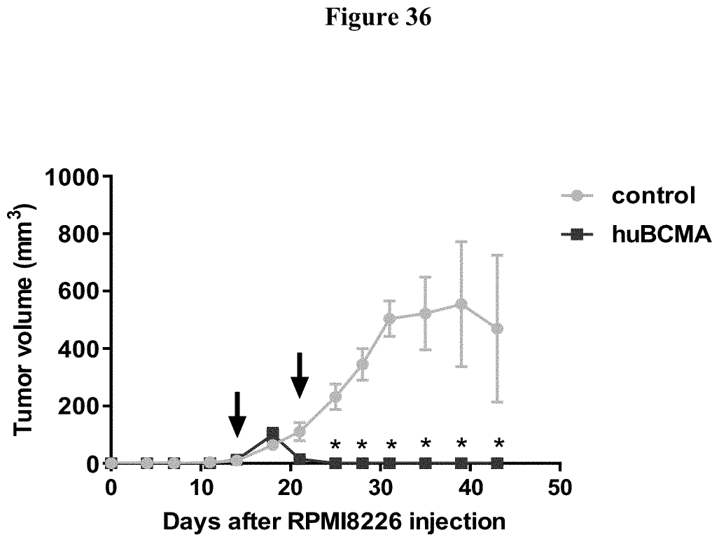

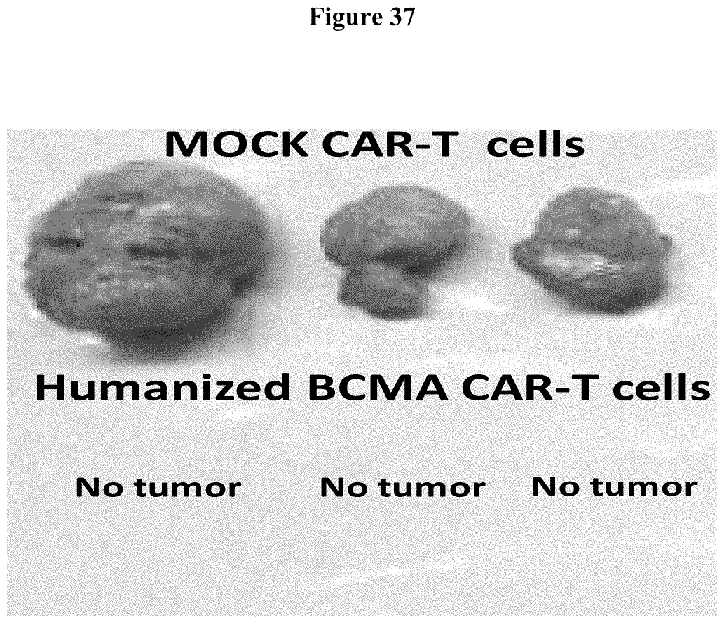

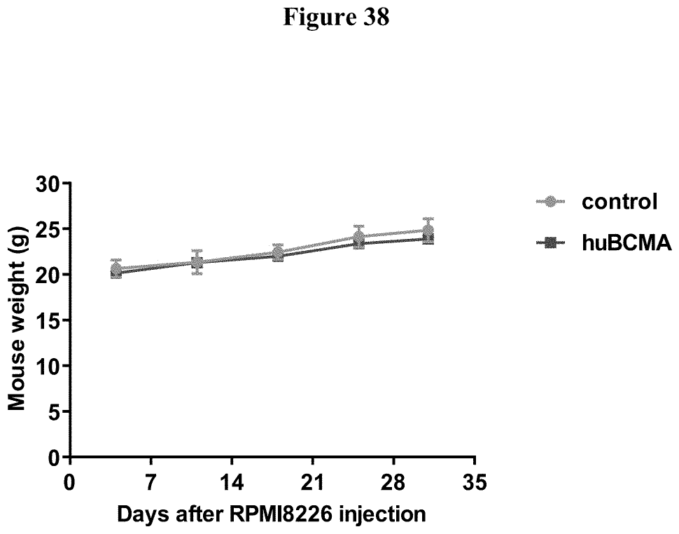

Applicants tested the humanized BCMA CAR-T cells in a xenograft tumor model, in which NSG immunodeficient mice develop subcutaneous tumors from BCMA-positive RPMI8226 multiple myeloma cells. Tumor size was measured biweekly for 6 weeks with calipers, and CAR-T cells or control T cells were administered intravenously on days 14 and 21. HuBCMA CAR-T cells completely blocked tumor growth (FIGS. 36 and 37) without affecting mouse health (inferred by mouse weight, FIG. 38). FIG. 36 describes the growth of RPMI8226 multiple myeloma tumors in mice. Tumor volumes were calculated from biweekly caliper measurements. FIG. 37 are photographs of the tumors at the end of the study. Cells were administered to the mice intravenously on days 14 and 21 (arrows). * p=0.0095 for hBCMA vs mock (Mann-Whitney test). FIG. 38 shows the weight of the mice in the tumor study. Mice were weighed weekly

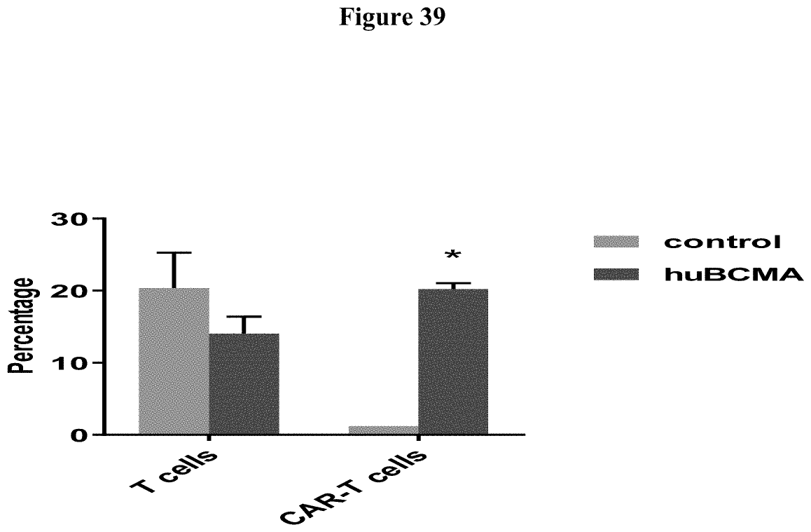

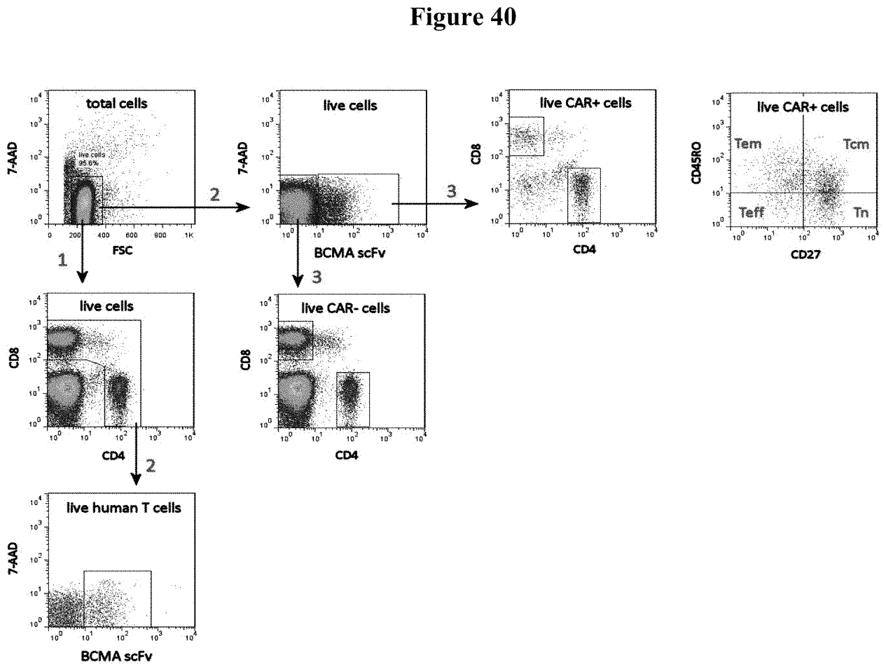

Flow cytometric analysis of the peripheral blood leukocytes at the end of the study indicated that approximately 15-20% of the cells were human T cells (FIG. 39, left). In the huBCMA CAR-T cell-treated mice, approximately 20% of the human T cells were CAR-T cells (FIG. 39, right). The gating and plotting scheme is shown in FIG. 40. FIG. 39 shows the frequencies of human T cells and BCMA CAR-T cells in mice. Peripheral blood leukocytes were stained with labeled BCMA protein, antibodies for human CD4 and CD8, and 7-AAD. The percentages of 7-AAD-negative cells that stained with either the CD4 or CD8 antibody (i.e. live human T cells) are shown on the left. The percentages of these T cells that bound to the BCMA protein (i.e., CAR-T cells) are shown on the right. * p<0.0001 for hBCMA vs control (1-way ANOVA with Tukey's post-hoc test).

In FIG. 40, there is shown the blood leukocytes first plotted for forward scatter (FSC) vs 7-AAD staining, and a gate (box) is drawn around the live (7-AAD-negative) cells. The gated live cells are then plotted for CD4 vs CD8 staining (arrow #1) and a new gate is drawn around the CD4⁺ cells and CD8⁺ cells (i.e., T cells). The gated live cells or live human T cells are then plotted (arrows #2) for binding to the BCMA protein (X-axis) vs 7-AAD. Finally, gated BCMA CAR⁺ cells and BCMA CAR⁻ cells are plotted (arrows #3) for CD4 vs CD8 staining and CD27 vs CD45RO staining.

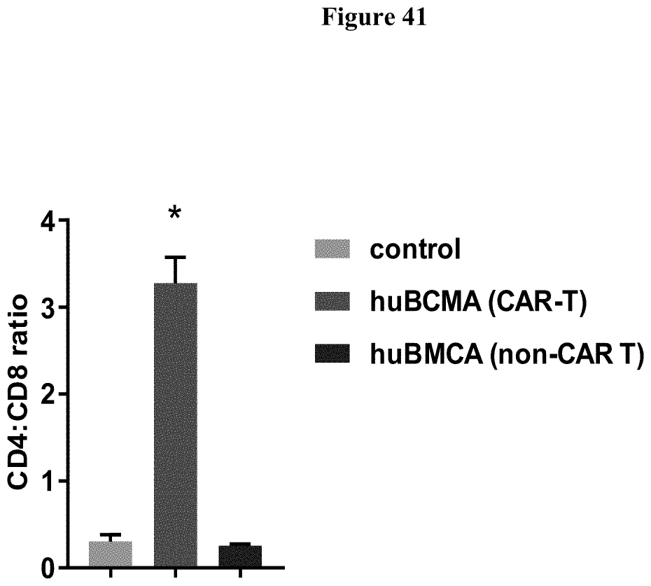

The CAR-T cells were analyzed for CD4:CD8 ratio and for differentiation status. BCMA CAR-T cells had a CD4:CD8 ratio of 3:1, indicating that they were mostly T-helper cells (FIG. 41). In contrast, the T cells in the mock-treated mice and the non-CAR T cells in the BCMA

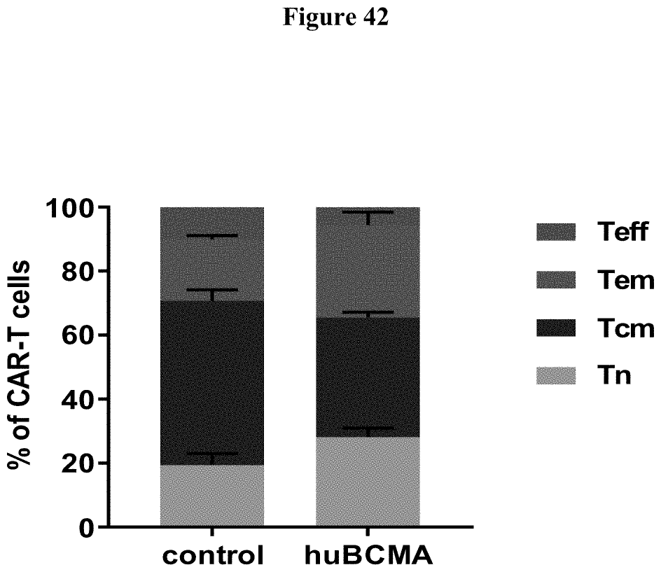

CAR-T cell-treated mice had a CD4:CD8 ratio of approximately 0.3:1, indicating that they were mostly cytotoxic T cells. Staining with CD27 and CD45RO antibodies indicated that the BCMA CAR-T cells comprised multiple subsets, but were primarily memory T cells (FIG. 42). Hence, unlike the non-CAR-T cells, the CAR-T cells were CD4+ memory T cells. FIG. 41 illustrates the CD4:CD8 ratios of the human cells in the mice. Peripheral blood leukocytes were stained with labeled BCMA protein, antibodies for human CD4 and CD8, and 7-AAD. Cells that did not stain with 7-AAD (i.e. live cells) were analyzed for BCMA protein binding and CD4 and CD8 expression. The CD4:CD8 ratio was calculated by dividing the frequency of CD4+CD8− cells by the frequency of CD8+CD4− cells.

FIG. 42 describes the differentiation status of the control T cells and huBCMA CAR-T cells in the mice. Left: peripheral blood leukocytes from control T cell-treated mice were stained with 7-AAD and antibodies for human CD3, CD27 and CD45RO. Cells that did not stain with 7-AAD but did bind to the CD3 Ab (i.e. live T cells) were gated and analyzed for CD27 and CD45RO expression. Right: peripheral blood leukocytes from huBCMA CAR-T cell-treated mice were stained with 7-AAD, labeled BCMA protein, and antibodies for human CD27 and CD45RO. Cells that did not stain with 7-AAD but did bind to the BCMA protein (i.e. live CAR-T cells) were gated and analyzed for CD27 and CD45RO expression. Tn (naïve T cells) are CD27+CD45RO−, Tcm (central memory T cells) are CD27+CD45RO+, Tem (effector-memory

T cells) are CD27−CD45RO+, and Teff (effector T cells) are CD27−CD45RO−.

### Example 15

### huBCMA CAR-T Toxicology

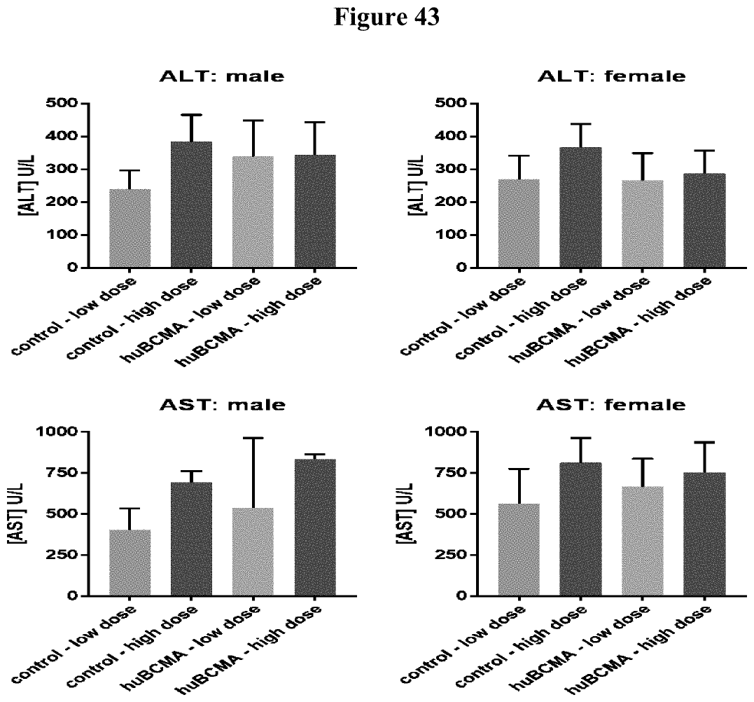

Twelve male and 12 female mice were injected intravenously with huBCMA CAR-T cells or control T cells at a high dose (2×107 cells/mouse) or low dose (2×106 cells/mouse). Three days later the levels of alanine aminotransferase (ALT) and aspartate aminotransferase (AST) in the blood were measured, and the heart, lungs, liver, kidney and colon were evaluated by a histopathologist. The levels of ALT and AST were similar between humanized BCMA CAR-T-treated mice and control T cell-treated mice at each dose (FIG. 43). In addition, the tissues of huBCMA CAR-T cell-treated mice showed no signs of pathological changes. FIG. 43 shows levels of toxicology markers ALT and AST in the blood of treated mice.

All literature and similar materials cited in this application including, but not limited to, patents, patent applications, articles, books, treatises, and interne web pages, regardless of the format of such literature and similar materials as well as all references cited in all of the above documentation, are expressly incorporated by reference in their entirety for any purpose as if they were entirely denoted. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

### REFERENCES

1. Maus, M. V., Haas, A. R., Beatty, G. L., Albelda, S. M., Levine, B. L., Liu, X., Zhao, Y., Kalos, M., and June, C. H. (2013). T cells expressing chimeric antigen receptors can cause anaphylaxis in humans. Cancer Immunol Res 1, 26-31.
2. Maus, M. V., Grupp, S. A., Porter, D. L., and June, C. H. (2014). Antibody-modified T cells: CARs take the front seat for hematologic malignancies. Blood 123, 2625-2635.
3. Ali, S. A., Shi, V., Maric, I., Wang, M., Stroncek, D. F., Rose, J. J., Brudno, J. N., Stetler-Stevenson, M., Feldman, S. A., Hansen, B. G., et al. (2016). T cells expressing an anti-B-cell maturation antigen chimeric antigen receptor cause remissions of multiple myeloma. Blood 128, 1688-1700.
4. Tai, Y. T., and Anderson, K. C. (2015). Targeting B-cell maturation antigen in multiple myeloma. Immunotherapy.
5. Boeye, A. (1986). Clonal isolation of hybridomas by manual single-cell isolation. Methods Enzymol 121, 332-340.

What is claimed is:

1. A single-chain variable fragment (scFv) of humanized BCMA comprising V_{H} having the amino acid of SEQ ID NO: 7, and V_{L} having the amino acid of SEQ ID NO: 8.

2. The scFv of claim 1, further comprises a linker in between V_{H} and V_{L}.

3. The scFv of claim 2, which has the amino acid sequence of SEQ ID NO: 6.

4. A chimeric antigen receptor (CAR) fusion protein comprising from N-terminus to C-terminus:
(i) The scFv of claim 3,
(ii) a transmembrane domain,
(iii) at least one co-stimulatory domains, and
(iv) an activating domain.

5. The CAR fusion protein of claim 4, which has the amino acid sequence of SEQ ID NO: 15.
